(19) United States
(12) Patent Application Publication
(10) Pub. No.: US 2007/0184515 A1
(43) Pub. Date: Aug. 9, 2007

(54) Method to determine 3-d elemental composition and structure of biological and organic materials via atom probe microscopy

(75) Inventors: Steven L. Goodman, Madison, WI (US); Thomas F. Kelly, Madison, WI (US); John J. Kremer, Cary, NC (US)

Correspondence Address: PERKINS COIE LLP;PATENT-SEA, P.O. BOX 1247, SEATTLE, WA, 98111-1247, US

(73) Assignee: Imago Scientific Instruments Corporation, Madison, WI (US)

(21) Appl. No.: 10/567,869

(22) PCT Filed: Aug. 6, 2004
(86) PCT No.: PCT/US04/25332

§ 371 Date: Nov. 15, 2006

Related U.S. Application Data
(60) Provisional application No. 60/492,789, filed on Aug. 6, 2003.

(51) Int. Cl. G01N 33/48

(52) U.S. Cl. 435/40.5

(57) ABSTRACT

Disclosed are methods to prepare a specimen for microanalysis, the specimens so prepared, and methods to analyze the specimens by atom probe microscopy. The specimens are prepared by embedding a specimen within an electrically conductive matrix to yield an embedded specimen; and then forming regions on the embedded specimen into shapes suitable for microanalysis by an atom probe.

### CROSS-REFERENCE TO RELATED APPLICATIONS

Priority is hereby claimed to provisional application Ser. No. 60/492,789, filed Aug. 6, 2003, the entire content of which is incorporated herein.

### FIELD OF THE INVENTION

The present invention relates to methods and instrumentation to determine the three-dimensional structure and elemental composition of biological materials at the molecular, near-atomic, and atomic level using three-dimensional atom probe microscopy and related methods.

### BACKGROUND

In the manufacture of many modem devices containing microscopically thin layers of different materials, and/or zones of different materials segregated on a microscopic scale, it is important to be able to study the different layers and/or zones with analytical equipment after the deposition. As examples, it is often useful to be able to microscopically analyze the structures of semiconductor microelectronic devices; magnetic thin film memory storage devices (such as read/write hard disk heads and platters); thin film-based optical devices; multilayered polymeric, organic and/or biochemical based thin film devices (as used in medicine); composites of inorganic materials, organic materials and/or biological materials (such as bioMEMs, biosensors, bioarray chips, and integrated labs on chips); and other devices wherein nanoscale structures are critical to device function. Common equipment used for such analysis (hereinafter referred to as “microanalysis”) includes electron microscopes (including TEMs, Transmission Electron Microscopes, and SEMs, Scanning Electron Microscopes); spectrometers (including Raman spectrometers and Auger spectrometers); photoelectron spectrometry (XPS); Secondary Ion Mass Spectrometry (SIMS); and more recently, the atom probe microscope, as described in U.S. Pat. Nos. 5,061,850 and 5,440,124, incorporated herein by reference. Of course, other microanalysis equipment is available, and new equipment having different principles of operation is expected to become available over time.

Of particular note is that detailed understanding of the atomic and molecular structure of biological molecules including proteins, nucleic acids, lipids, polysaccharides, and other components is at the root of much contemporary biology, medicine, and biotechnology. This is because the three-dimensional (3-D) atomic and molecular structure of a compound is what ultimately dictates its biomolecular function. For example, the 3-D structure of proteins dictates the activity of enzymes, the specificity of receptors and antibodies, and how proteins (and other biomolecules) interact with each other. Targeting drugs to interact specifically with certain proteins, and/or to interact with particular ligands or epitopes on these proteins, is similarly also dependent upon the atomic and molecular structure of the target protein. The structure of DNA and RNA is of similar importance in determining function, drug targeting, and understanding of biological processes. Because of the importance of the 3-D structure of a proteins and nucleic acids, there has been considerable effort to develop methods to determine these structures. For a detailed understanding of structure-function relationships, near-atomic structural resolution of a few angstroms (tenths of nanometers) is required. Conventional methods to achieve this resolution with bio-macromolecular and organic structures can be roughly categorized into the following groups:

(1) Methods that utilize computational modeling to predict 3-D protein or nucleic acid structure based upon knowledge of the primary sequence of amino acids and base pairs, respectively. Similar methods may also be applied to other organic materials.

(2) Methods that utilize X-ray diffraction through crystals of protein, nucleic acid, or other molecules. This group of methods includes closely related methods utilizing the diffraction of other electromagnetic waves and particles (e.g., electrons, neutrons).

(3) Methods that utilize nuclear magnetic resonance (e.g., ¹H-NMR, ¹³C-NMR, and the 2-D protocols COSY, ROESY, NOESY, and the like) to determine the proximity of chemical moieties to each other.

(4) Methods based upon high-resolution microscopies, including conventional electron microscopy, scanning tunneling microscopy, and atomic force microscopy.

Each of these methods has certain inherent limitations, especially when applied to biologically important molecules:

Computational Modeling: The major difficulty for computationally determining 3-D structures based upon knowledge of the primary amino acid sequence of a protein is that these require massive calculations. A small protein of only 100 amino acid residues has an astronomical number of possible conformations, on the order of 10¹⁶ (see K. A. Dill, Biochemistry, 24, 1501-1509, 1985). Moreover, the chemical and mathematical rules to choose the proper conformation are not entirely known. In fact, because proteins exist in a dynamic equilibrium with water, salts, other proteins, and various other biomolecules, the biological conformation of any given protein may not be the lowest energy state, but rather some unknown, higher energy state. Instead of calculating conformations de novo, some computational methodologies utilize sequence homologies. In these methods, proteins containing similar sequences and having a known conformation are used as a basis to calculate the structure of the unknown protein. However, this assumes that the conformation of the known protein portion will be mirrored in the unknown protein. Such an assumption may not be warranted. Moreover, computational modeling requires that there be known structural homologies; therefore these structures must be determined by some other means. Finally, computational methods for determining protein structure from amino acid sequences cannot be generally extended to determine the conformation of other biomolecules such as nucleotides, nucleoproteins, carbohydrates, glycosaminoglycans, and proteoglycans. Nor can models designed to predict protein 3-D structure be extended to predict the 3-D structure of non-biological organic materials such as synthetic polymers.

X-Ray Crystallography: Methods based upon X-ray crystallography regularly provide the highest resolution of current methods, on the order of 2-3 angstroms (i.e., atomic-level resolution) for proteins, nucleic acids, synthetic polymers, and many other materials. However, obtaining such information is a very slow and laborious process. It can take months to convert X-ray crystallographic data into a corresponding 3-D structure because diffraction patterns are extremely complex (e.g., as many as 25,000 diffraction spots from a single small protein). Solving a diffraction pattern has been described as directly analogous to reconstructing the shape of rock from the ripples it creates when thrown into a pond (see: Molecular Cell Biology, Lodish et al, Scientific American Books, New York, 1995). Additional difficulties in crystallographic analysis arise because the molecule under study must first be crystallized into a highly regular crystal with millimeter or near-millimeter dimensions. This requires that the molecule under study must be removed from its normal milieu or produced in quantity by various means, must be highly concentrated, and then must be crystallized. The single act of preparing a suitable crystal to begin the analysis can itself take months, years, or even prove impossible. In roughly 50 years of continuous effort, less than two percent of the over 100,000 known different proteins have been grown as crystals suitable for X-ray diffraction studies. See, for example, U.S. Pat. No. 5,597,457, “System and Method for Forming Synthetic Protein Crystals to Determine the Conformational Structure by Crystallography,” issued Jan. 28, 1997, to Craig et al.

Further still, the resolution obtained is limited by the quality of the crystal analyzed. Due to the time required for crystallization and the collection and analysis of the diffraction data, determining the structure of a single, relatively simple macromolecule can easily take a year or more. And, as noted in the prior paragraph, a number of biologically important compounds have so far proven to be impossible to crystallize, including certain membrane proteins, pharmacologically important receptors, and macromolecular complexes.

Nuclear Magnetic Resonance: High-resolution nuclear magnetic resonance (NMR) can provide angstrom-level resolution comparable to X-ray crystallography on many types of biological and organic samples. Like crystallography, NMR also requires separation and purification. Hence the molecule of interest must be isolated from its normal environment thereby potentially altering its conformation. The higher resolution method of liquid state NMR (in contrast to solid state NMR) is generally limited to molecules of a molecular weight no larger than about 40 kD due to the requirement that the molecules rapidly tumble in solution. Some recent reports suggest that the latest generation high-field NMR magnets operating at 800-900 MHz, may enable the determination of structures up to 100 kD. Nonetheless, high-resolution NMR requires that the biomolecule must be removed from its natural environment and then solubilized. In addition, protein samples for NMR are often isotopically labeled to determine the relative locations of particular chemical moieties. The labeling process provides yet another manipulation that can alter structure.

High-resolution NMR is not able to analyze high-molecular weight (MW) biomolecules, such as many larger proteins and macromolecular complexes. Although solid-state NMR does not have the size and solubilization limitations of liquid-state NMR, the lower resolution limitation of solid-state NMR is only able to provide nanometer-level resolution, and not the necessary angstrom-level resolution. Finally, the analysis of NMR spectra is complex, time-consuming, and less than straightforward.

High-Resolution Microscopy: Current scanning and transmission electron microscope technology is not able to provide the necessary sub-nanometer resolution with biological and organic materials. While high-resolution transmission electron microscopies (e.g., TEM, intermediate- and high-voltage transmission electron microscopy [IVEM and HVEM], scanning transmission electron microscopy [STEM], and scanning electron microscopies [SEM]) can provide instrumental resolution in the angstrom range with some robust materials (e.g., metals and ceramics), this is not possible with organic materials because low atomic number organic elements provide low intrinsic contrast and are easily destroyed by electron beams. In practice, the obtainable resolution for most biological and organic materials is, at best, in the 1-5 nm range, and more often in the 5-10 nm range. This is 10-to 50-fold poorer resolution than the required for atomiclevel resolution. Higher resolution electron imaging in the 1-3 nm range and potentially higher can in some cases be achieved by averaging multiple images of identical and ideally dimensionally regular structures such as certain macromolecular complexes or viruses. However, angstrom-level resolution has not been achieved for biological materials using any type of electron microscopy.

Moreover, the images that are obtained are averaged structures, computed from multiple imaging runs. Consequently individual details may be averaged out.

Finally, electron microscopy does not directly provide compositional or elemental information. These are generally provided by indirect radiative emission methods such as energy dispersive X-ray microanalysis (EDX), wavelength dispersive X-ray microanalysis (WDS), and by other methods such as electron energy loss spectroscopy (EELS) and related techniques. With carbon and the other low atomic number elements found in biological and other organic materials, the obtainable spatial resolution for elemental mapping is rarely better than about 10 nanometers and provides limited sensitivity.

Scanning probe microscopies, including atomic force microscopy (AFM), scanning tunneling micrscopy (STM), near-field scanning optical microscopy (NSOM), and related methods, are also not suitable for providing 3-D atomic resolution and elemental mapping of biological and organic materials. While atomic spatial resolution is possible using these devices, this class of devices can probe only the surface of a sample. They are not capable of gathering information on the layers underlying the surface. Scanning probe microscopies also do not provide information on elemental composition.

A promising technology, local electrode atom probe (LEAP) microscopy is described in U.S. Pat. Nos. 6,576,900 and 6,700,121, both of which are incorporated herein. As described in these two patents, atom probes have conventionally utilized a needle-shaped study specimen (or a study specimen having a needle-shaped study region formed thereon) because such a needle shape is beneficial for creating the high electric fields required for atom probe microanalysis. See FIG. 1A which illustrates that shape and millimeter-sized scale of conventional atom probes. Where the study specimen or study region is wire-shaped, this shape readily lends itself to needle creation; otherwise, the region to be studied must be cut into a suitable needle-like shape, via, for example, focused ion beam (FIB) milling. Planar structures like wafer-processed materials, e.g., microelectronic materials, are often difficult to cut into atom probe specimens because the structures of interest exist only in a very thin layer on the surface of the specimen, a specimen that is often less than about 10 micrometers (microns) thick. However, with advances in atom probe technology, and with the advent of scanning atom probes and local electrode atom probes, it is possible to use atom probes to microanalyze specimens that are raised in relation to their surroundings by as little as a few micrometers, and which are closely spaced (e.g., by no more than a few micrometers away) in relation to adjacent protrusions. For example, local electron atom probes only require a small protrusion on the specimen (a few microns high) for the local electrode to be able to locally apply the necessary extraction field to the specimen in order to effect ionization. In the conventional approach, however, the specimen itself must have sufficient electrical conductivity in order to be analyzed via atom probe microscopy.

Thus, as described in the U.S. Pat. Nos. 6,576,900 and 6,700,121 patents, a study specimen is formed from a larger first study object such as an integrated circuit wafer, as by cutting the study specimen therefrom by the use of FIB milling. The study specimen is usually a portion of the first study object item which is of key interest for microanalysis, e.g., a functional section of a semiconductor chip. The study specimen is then removed from the first study object, and is situated on a second study object such as a silicon-based wafer whereupon the study specimen is microanalyzed. Preferably, the study specimen (and perhaps several other study specimens) are also inserted within recesses in the second study object, and/or are affixed to the second study object (as by FIB deposition). The study specimen(s) can then be microanalyzed on the second study object, which can be constructed and configured to enhance the speed and ease of microanalysis. For example, the second study object may be formed of a material which promotes electrostatic attraction of the study specimen to the second study object (either by itself or with the assistance of an applied charge), thereby assisting in the placement of the study specimen on the second study object.

The sample preparation techniques described in U.S. Pat. Nos. 6,576,900 and 6,700,121, however, are not suitable for analyzing biological materials, organic materials, and otherwise non-conductive materials.

As a consequence, there remains a long-felt and unmet need in the scientific community for a fast, reliable, and robust method to determine the 3-D structure and composition of biological and organic materials at atomic and/or near-atomic resolution.

### SUMMARY OF THE INVENTION

This invention is made practical by the development of improved 3-D atom probe microscopes, as exemplified by Imago Scientific Instruments' local electrode atom probe (LEAP). Such improvements include substantial increases in the microscope field of view, the geometry of the specimens that may be examined, and much more rapid analysis times. Nonetheless, the invention described herein can be applied to conventional atom probes by those skilled in the art Thus, the subject invention also pertains to determining the structure of biological and organic materials with other types of atom probes and related instruments.

The methods and instruments described herein can determine the structural and compositional analysis of biological and organic materials, including (without: limitation) proteins, lipids, carbohydrates, and nucleic acids, as well as biomolecular and biomacromolecular assemblies such as receptor complexes, receptors coupled with ligands, enzyme-substrate complexes, drug-target complexes, membranes, membrane-bound proteins, cellular organelles, whole cells, and other biological components, including tissue specimens.

This invention also relates to methods and instruments to determine the 3-D structure and elemental composition of synthetic and natural organic materials at the molecular, near-atomic, and atomic level. This includes organic materials, coatings and thin films, polymers, polymeric and organic composite materials, liquid crystals, and both synthetic and natural materials that may contain multiple organic materials in association with inorganic materials. Explicitly included within the specimens that can be examined via the present method are (without limitation), biological materials such as proteins, nucleic acids, biomacromolecules, biomacromolecular complexes, and viruses; organic compounds such as intrinsically conductive polymers and other polymers that have been made suitably conductive via chemical treatment (as by treating with osmium or ruthenium compounds, see below); and non-conductive materials embedded within a suitably conductive matrix, such as organic nano-particles (e.g., dendrimers, polymers, fullerenes, and the like), inorganic nano-particles (e.g., ceramics, dielectrics, colloids, and micro- and nano-particulate materials), and nano-porous and micro-porous catalysts, zeolites, and other materials having nano-scale or micro-scale voids and cavities.

One particular area of application of the present invention is to determine the 3-D structure and elemental composition of proteins, polynucleic acids, and oligonucleotides. This information is highly prized in proteomics, genomics and pharmaceutical research and development.

A second particular area of application of the present invention is to determine the nanostructure and elemental composition of proteins, macromolecular complexes or other structures that are located within biological tissues, cellular components, cellular organelles, extracellular organelles, viruses, bacteria, other micro-organisms, or other biological systems or components.

A third particular area of application is to determine the nanostructure and elemental composition of man-made or partially man-made biological structures, including tissue engineering scaffolds, cell culture systems, and other biological-synthetic constructs.

A fourth particular area of application of the present invention is to determine the nanostructure of polymers, polymer coatings, and other coatings that may contain organic components.

A fifth particular area of application is to determine the nanostructure of nano—and micro-scale biotechnological devices such as bio-array chips, biosensors, biomaterials, and bioMEMs.

A sixth particular area of application includes determination of the nanostructure of materials that may not be organic or biological, but that may be prepared and analyzed using means that are similar to those described herein. Such materials include fullerenes, ceramics, dielectrics, nano—and micro-porous catalysts, zeolites, materials with nanoscale voids and cavities, colloids, and micro and nano—particulate materials.

Thus, a first embodiment of the invention is directed to a method of preparing a specimen for microanalysis. The method comprises embedding the specimen within an electrically conductive matrix to yield an embedded specimen and forming regions on the embedded specimen into shapes suitable for microanalysis by an atom probe. It is preferred that the specimen be embedded within a polymer matrix, more preferably still an intrinsically conductive polymer matrix. The polymer matrix may optionally be treated to increase its conductivity, as by treating the polymer with a metal-containing compound (preferably an osmium-containing compound or a ruthenium-containing compound, and more preferably still osmium tetroxide or ruthenium tetroxide). The specimen can also be embedded within a hydrogel.

The specimen can be embedded within the matrix by a number of different routes. For example, the specimen can be embedded within the electrically conductive matrix by mixing the specimen with a corresponding monomeric compound and then polymerizing the monomeric compound to yield the electrically conductive matrix. Or the specimen can be embedded within the electrically conductive matrix by mixing the specimen with a corresponding pre-polymer compound and then polymerizing the pre-polymer compound to yield the electrically conductive matrix. Alternatively, the specimen can be embedded within the electrically conductive matrix by mixing the specimen with a corresponding water-soluble monomeric compound and then polymerizing the monomeric compound in aqueous solution to yield the electrically conductive matrix. This specific embedding step is beneficial for water-soluble specimens because the specimen can be embedded directly, without removing water.

In the preferred embodiment, the specimen is embedded within a matrix comprising an intrisically conductive polymer (ICP), preferably an ICP comprising polymers and/or copolymers containing polythiophenes, polyanilines, polypyrroles, and combinations thereof.

The embedded specimen can also be disposed on a substrate, either prior to forming the embedded specimen or after forming.

The regions suitable for microanalysis by an atom probe are preferably formed using focused ion beam lithography, although any suitable method for forming an atom probe (now known or developed in the future, such as mechanical micro-machining, magnetron sputtering, chemical etching, photo-etching, and the like) can be used. in one preferred route, the regions are formed by doping the embedded specimen with a masking agent and then exposing the embedded specimen to a broad ion beam under conditions and for a time sufficient to remove the masking agent from the embedded specimen. In this fashion, regions protruding from embedded specimen and suitable for microanalysis by an atom probe are formed. (The protruding regions correspond to those regions initially masked by the masking agent.) Alternatively, the embedded specimen can be disposed on a substrate prior to forming, to yield a specimen-coated substrate. Then, regions on the specimen-coated substrate suitable for microanalysis by an atom probe are formed. It is preferred that the regions are formed using focused ion beam lithography.

As noted earlier, the regions can also be formed by doping the specimen-coated substrate with a masking agent and then exposing the specimen-coated substrate to a broad ion beam under conditions and for a time sufficient to remove the masking agent from the embedded specimen, whereby regions protruding from embedded specimen and suitable for microanalysis by an atom probe are formed.

In the preferred embodiment, an organic or biological specimen is embedded within the matrix.

The embedded specimen may also be disposed on a pre-formed atom probe. Here, regions suitable for microanalysis by an atom probe are formed on a substrate. The embedded specimen is then immobilized on the pre formed regions of the substrate. In this fashion, regions on the embedded specimen are formed into shapes suitable for microanalysis with an atom probe.

Alternatively, regions suitable for microanalysis by an atom probe are formed on the substrate and the specimen is immobilized on the formed regions of the substrate (either directly of via the use of linking moieties). The formed regions of the substrate, with the specimen immobilized thereon, are then coated with the electrically conductive matrix, whereby the specimen is embedded within the matrix.

The specimens prepared as described herein can then be microanalyzed, preferably by atom probe microscopy, and more preferably still by local electrode atom probe microscopy.

The invention also encompasses an atom probe fabricated by any of the methods disclosed herein.

### BRIEF DESCRIPTION OF THE DRAWINGS

FIG. 1A is a schematic of the PRIOR ART illustrating the required sample geometry for PRIOR ART atom probes. FIG. 1B is a schematic illustrating the preferred sample geometries for use in the present invention.

FIG. 2 is a stylized rendering depicting a stabilized protein or other polymeric specimen prepared for atom probe analysis according to the present invention. Covalent cross-links are shown as broken lines. Thin, solid lines indicate bonds linking the polymeric specimen to the substrate.

FIG. 3 is a flowchart-for preparing and imaging biological and organic materials with a local electrode atom probe (LEAP) according to the present invention.

FIG. 4 is a stylized rendering illustrating the en bloc embedding of a biomolecular specimen (a protein) and an SEM micrograph of an actual embedded specimen.

FIG. 5 is a photomicrograph showing a microtip of CPUD2-brand intrinsically conductive polymer (ICP) disposed on a bulk specimen of CPUD2 polymer. The microtip was prepared with a masking partide, followed by broad ion beam (BIB) etching, as per the present invention.

FIGS. 6a to 6h taken together schematically illustrate various methods to prepare specimens according to the present invention. FIGS. 6a and 6b illustrate the rough molding of a specimen disposed within an ICP embedment matrix. FIG. 6b illustrates the rough-shaped, embedded specimen. FIG. 6d illustrates the sharpening of the embedded specimen with a FIB. FIG. 6e . illustrates a pre-formed tip of metal or conductive silicon prior to depositing a thin-film layer of a specimen dispersed in an embedding matrix. FIG. 6f illustrates the tip shown in FIG. 6e after deposition of a thin film of embedded specimen. FIG. 6g illustrates a similar tip having an intermediate layer of adhesive, such as a self-assembled monolayer (SAM) of thiols (or other chemistries) to assist in adhering the embedded specimen to the tip. FIG. 6h illustrates the tip as shown in FIG. 6g with a layer of specimen molecules (the spheres) immobilized to the SAM layer and further having a layer of embedding matrix disposed on top of the immobilized specimen.

FIG. 7 is a SEM photomicrograph depicting a ˜150 nm diameter atom probe tip prepared from Baytron CPUD2-brand ICP.

FIG. 8 is a schematic depicting two methods to fabricate LEAP tips from specimens encapsulated in a block of ICP.

FIG. 9 is a flow chart of schematic diagrams depicting alternative routes for preparing specimens according to the present invention.

FIGS. 10A-10E are SEM images of various steps in the production of atom probe or LEAP specimens of ICP on silicon, so as to embed biological or organic specimens on a silicon surface. FIG. 10A is a cross-section image showing a uniform 0.5 μm thickness ICP coating. FIG. 10B is a low-magnification view of the ICP-coated silicon wafer of FIG. 10A after dicing to produce approximately 100 protruding posts topped by an ICP coating. FIG. 10C is a higher-resolution image of.FIG. 10B showing two diced posts; this figures shows that the ICP coating is intact in the entire center region of each post, with only minimal detachment of the ICP coating at the edges of each post. (This minor chipping is not a problem because LEAP analysis occurs only in the center of the post.) FIG. 10D is an image of the end of a single post after FIB sharpening. FIG. 10E is an image depicting the very tip of the sharpened post. The intact ICP tip is the slightly hazy layer on the uppermost end of the tip.

FIG. 11 is a flow chart depicting specimen preparation steps for en bloc-prepared specimens and pre-sharpened tips. Optional steps are depicted by boxes drawn in broken lines.

FIG. 12 is a mass spectrum of graphite obtained with LEAP.

FIG. 13 is a mass spectrum of pyrolytic carbon that was manufactured for use a heart valve prosthetic.

FIGS. 14A-14C are LEAP mass spectra of osmium-treated CPUD2-brand ICP specimens. The large peak in FIG. 14A (the full-width mass spectrum) appears to be copper, and is not part of the CPUD2 itself. (Copper was used in the specimen support and may be present in the sample due to indirect sputtering during FIB sharpening.) FIG. 14B is a higher-resolution spectrum of the same spectrum as FIG. 14A. FIG. 14B shows multiple carbon peaks. FIG. 14C is a mass spectrum for a second specimen (Specimen B). The mass spectrum shown in FIG. 14C includes peak assignments for fragments of the ICP.

FIG. 15 is a LEAP 3-D image reconstruction of osmium-treated CPUD2-brand ICP whose mass spectrum is depicted in FIGS. 14A and 14B. Most masses with clear peaks are shown. The larger dots represent the larger masses, while the smaller dots represent the smaller masses. The mass peak representing Cu is not shown.

FIG. 16 is a cross-sectional portion of the 3-D image reconstruction shown in FIG. 15. Only the two large masses are shown. These presumably correspond to the phenyl and sulfonate groups of the poly(styrene sulfonate) block component of the CPUD2 ICP.

FIGS. 17A-17C are LEAP mass spectra for two identical specimens of ICP cast onto a silicon substrate. FIG. 17A is the spectrum for specimen 1; FIG. 17B is the spectrum for specimen 2. FIG. 17C is a repeat spectrum for specimen 2 with a slightly altered alignment of the local electrode. Note that all three spectra are quite similar.

FIGS. 18A and 18B are LEAP mass spectra for the same ICP formulation cast on a silicon substrate as in FIGS. 17A-17C. The entire mass spectrum is depicted in two sub-spectra to facilitate peak analysis. FIG. 18A spans atomic mass from 0 to 35; FIG. 18B spans atomic mass from about 30 to about 75. Tentative assignments of molecular fragments for several peaks are shown.

FIG. 19 is a 3-D reconstruction of the ICP spectra shown in FIGS. 18A and 18B. The filled black circles are carbon, grey circles are sulfur, white circles are oxygen. For clarity, all atoms in the specimen are not shown. The axis dimensions are in nanometers. The region shown is approximately 30 nm in diameter and 5 nm thick.

FIG. 20 is a flow chart and corresponding schematics illustrating the software protocol for analyzing proteins or other polymeric materials according to the present invention.

### DETAILED DESCRIPTION OF THE INVENTION

(1) Overview:

The vast majority of specimens previously analyzed using atom probes have been metals. The reasons for this are two-fold: 1) the need for the specimen to have high electrical conductivity; and 2) the need for the specimen to have sufficient physical strength to be shaped into a millimeter-long, needle-shaped specimen (the required geometry). Consequently, there is only a modest history of atom probe analysis of biological or organic specimens. Field ion microscopy (FIM) images of freeze-dried tRNA dimers adsorbed onto iridium tips were reported by Machlin in 1975 (Machlin, Freilich et al. 1975), while Panitz has provided what appears to be the most recent report of FIM and field desorption imaging, which was applied to ferritin (Panitz 1981; Panitz 1982) and unstained DNA (Panitz 1983). Other than a few additional publications by these research groups, this is essentially the entire history of biological atom probe and FIM imaging.

A major problem discussed in some detail by Machlin et al. is that the high field strengths for FIM imaging (˜5-15 V/nm) appeared to cause desorption and fragmentation of adsorbed biomolecules. (Machlin, Freilich et al. 1975). While Machlin et al. saw macromolecular desorption or fragmentation as a problem, the present inventors view this phenomenon as an opportunity. By accelerating the ionized macromolecular fragments to a two-dimensional microchannel plate detector, both positional and molecular weight information can be obtained. Thus, rather than hampering the characterization of biomolecules, the present inventors set about to show that atom probes can be made to harness the ionization phenomenon to determine both the 3D structure and the atomic or molecular composition of the probe. Such determination is highly useful in structural biology, biomedical science, biomolecular engineering and related disciplines. Because it is axiomatic that structure begets function, elucidating the 3-D structure of a biomolecule (at the atomic or molecular level) is a critical key to elucidating its function.

There have also been several reports that utilized atom probes to examine non-biological organic materials. Nishikawa examined diamond-like carbons with a non-imaging (i.e., one-dimensional) atom probe to obtain compositional depth profiles (Nishikawa, Sekine et al. 1997; Nishikawa, Sekine et al. 1998). Other investigators used FIM and atom probes to examine graphite fibers (Liu and Tsong 1988), the intrinsically conductive polymer (ICP) polypyrrole (Maruyama, Hasegawa et al. 1987), and phthalocyanine (Iwatsu, Morikawa et al. 1987).

Although this history is limited, these reports indicate the potential to use atom probes to analyze conductive carbon-based materials. However, the masses of ionized fragments in these reports were considerably larger than one or a few atoms. The masses in the prior work were relatively large molecular fragments of five to eight (or more) atoms in size (exclusive of hydrogen). Moreover, many ions were small polymers consisting of two or more repeat units. Such large fragment and small polymers are not desirable for determining the structure of macromolecules with an atom probe because the size of an ionized fragment determines the spatial resolution achievable. Thus, spatial resolution is decreased with large ionized molecular fragments. In addition, determining the composition of larger fragments requires extremely sensitive mass resolution.

Therefore, for ideal atom probe determination of biomolecular structures, the ionized fragments should be individual atoms or very small molecular fragments, preferably on the order of one to four atoms, exclusive of hydrogen. Larger ionized molecular fragments that have a known and determinable 3-D conformation, and that are small in size, will not significantly decrease the use of the atom probe for structural analysis of macromolecules. Examples of such moieties include phenyl groups and other ring structures, and many pendant moieties such as sulfonates and methyls.

Due to advances in atom probe instrumentation, especially as exemplified by Imago's LEAP microscope (Kelly, Camus et al. 1995; Kelly and Larson 2000), atom probe imaging of biological and organic materials (and many other materials as previously addressed) becomes practical. These advances include the LEAP's much larger field of view of circa 100 nm which is substantially larger than conventional atom probes that generally provide a field of view less than 10-20 nm. Because biomolecules and macromolecular complexes are typically 10 to 100 nm or larger, a 10-20 nm field of view is generally inadequate. The LEAP also provides a much faster data acquisition speed of ˜10,000 ions/second, in comparison to circa 10 to 100 ions/second in comparison to other atom probe designs. This additional speed greatly accelerates analyses that might have required days or weeks with other atom probes to now be achieved in minutes or hours with the LEAP. Finally, the LEAP's local electrode design provides substantial benefits with respect to the geometry of specimens that may be analyzed. Specimens need not be 5 mm long needles that in turn must be mechanically sturdy and have a high level of electrical conductivity. In the present invention, the specimens need only be a few tens of microns in length (preferably about 100 μm or less, more preferably about 50 μm or less, and more preferably still about 20 μm or less in length). This enables less sturdy materials such as polymers to be analyzed. Further, because the conductive path is much shorter, lower conductivity materials that cannot be analyzed by conventional methods can be analyzed using the present invention.

The processes, methods and instruments described herein, while explicitly developed for LEAP analysis, can also be used to prepare specimens for other atom probes and related instruments (all such related processes being referred to herein as “microanalysis”).

(2) Local Electrode Atom Probe (LEAP) Analysis:

The local electrode (or scanning) atom probe was initially envisaged by Nishikawa for analyzing planar specimens. See O. Nishikawa and M. Kimoto, Appl. Surf Sci., 74/75 (1994) 424; and T. F. Kelly and D. J. Larson, Mate.r Characterization, 44 (2000) 59. The original intention was for the characterization of naturally occurring protrusions or microtips fabricated from the surface layers of a specimen by focused ion beam techniques. However, the instrument may also be applied to traditional needle-shaped field ion microscopy specimens.

In LEAP analysis, three-dimensional images of the internal structures of the specimen are generated from the acquired data set (mass/charge and time). The atomic coordinates and the mass-to-charge ratio are determined for each ion collected in a time-of-flight mass spectrometer that is equipped with a position-sensitive single-atom detector. Briefly, in a LEAP device, a funnel-shaped electrode (the local electrode) is positioned in close proximity to the tip of a needle-shaped specimen. Atoms are removed from the specimen in a controlled fashion by applying a high-voltage pulse to the local electrode. Disposed behind the funnel-shaped local electrode (and synchronized with the electrode) is a time-of-flight mass spectrometer (TOF-MS). Thus, atoms and fragments ionized from the specimen surface are accelerated through the electric field created by the funnel-shaped local electrode, and pass into the TOF-MS, where the mass-to-charge ratio (m/z) of each fragment is determined. In short, a high-voltage pulse sequence is repetitively applied to the field-defining local electrode in front of the specimen (which is often cryogenically cooled) to field evaporate ions from the surface of the specimen. These ions are then immediately analyzed by TOF-MS.

The major difference between the local electrode atom probe and earlier three-dimensional atom probes is the diameter of the aperture in the counter electrode and the proximity of the electrode to the apex of the needle-shaped specimen. In a conventional three-dimensional atom probe, a 5- to 10-mm diameter aperture is typically placed about 4 tol 0 mm in front of the specimen. In contrast, in conventional LEAP analysis, a 20- to 50-μm diameter aperture is typically positioned approximately one aperture diameter in front of the specimen. As noted earlier, in conventional LEAP, the needle-shaped specimen is usually several millimeters long. In order to align the specimen with the aperture in the funnel-shaped electrode, the specimen is mounted on a three-axis nanopositioning stage. This stage may also be used to scan across the surface of a planar specimen to locate protruding microtips, etc. The advantage of the LEAP configuration is that a significantly lower voltage is required to produce the field strength required to field evaporate ions from the specimen. For example, as compared to the conventional three-dimensional atom probe, about 50% of the voltage is required for a 30 μm aperture positioned roughly 10 μm in front of the apex of the specimen. Because the applied voltages are lower, it is economical to build voltage pulsers that can operate at orders of magnitude faster pulse repetition rates of up to 200 kHz. These pulsers, coupled with crossed delay line detectors, enable significantly faster data acquisition rates to be achieved with LEAP. Due to the close proximity of the local electrode to the specimen, the transit times of the ions to the electrode and the field-free region of the mass spectrometer are considerably shortened, thereby eliminating the major source of energy deficits that degrade the mass resolution. As reflectrons are not necessary, a larger field of view of the specimen may be analyzed. For a more detailed description of LEAP analysis, see, for example, M. K. Miller, “Atom Probe Tomography,” Kluwer Academic/Plenum Press, New York, © 2000.

(3) Sample Preparation:

The LEAP and other atom probes are high vacuum instruments. Therefore, specimens that are normally hydrated, such as biological cells or tissues (which are roughly 70% water by weight) must be carefully prepared in order to prevent rapid dehydration and collapse in the vacuum. Many of the sample preparation methods used for other high vacuum analytical instruments, such as electron microscopy, provide a starting point for such preparation. However, the special attributes and capabilities for atom probe and LEAP analysis require several different considerations, as discussed below for different types of organic and biological specimens. Sample preparation protocols can be separated into those that require rapid freezing (cryogenic protocols) and those that do not require freezing. In contrast, many specimens that are not hydrated, or otherwise solvated, may be prepared without any special handling to remove water (or another solvent).

To examine biological materials according to the present invention (and using the LEAP or other atom probes), several conditions must be met with respect to specimen preparation. These conditions are discussed in Table 1.

TABLE 1
Specimen preparation conditions for atom probe
analysis of biological and organic materials:

| | |
|---|---|
| 1. | Native structure must be maintained in the vacuum environment prior |
| | to and during analysis. |
| 2. | Biological materials must be immobilized and stable in the high |
| | electric field of the atom probe (circa 3-15 V/nm). |
| 3. | Biological materials must field ionize from the surface of the |
| | specimen in a controlled manner as single atoms or small |
| | molecular fragments. |
| 4. | The specimen must be prepared with a needle-like geometry wherein |
| | the end of the needle must have a radius of no more than a few |
| | hundred nanometers, and generally about 50 nm. |
| 5. | The end of the needle must form a uniformly curved and smooth |
| | surface in the electric field, and this must be maintained during |
| | analysis. |
| 6. | The material must have a sufficient level of electrical conductivity. |

The conditions discussed in Table 1 may be addressed as follows:

Condition 1—maintain native structure: Maintaining native structure can be achieved by using methods similar to those for preserving native (or near native) structures for transmission electron microscopy (TEM) and/or scanning electron microscopy (SEM) analysis. It is important to recognize that TEM specimen preparation methods are able to provide structural information at near-atomic resolution for biological materials (Baker, Olson et al. 1999; Baumeister and Steven 2000; van Heel, Gowen et al. 2000). The primary limitation for electron microscopy resolution of biological materials is not due to specimen preparation concerns, but rather is due to destructive interactions of the electron beam with the specimen. Most biological materials simply will not tolerate the electron flux required to generate a high resolution electron micrograph. In TEM this limitation is overcome by averaging dozens of low-electron-flux, low-contrast images. In direct contrast, when using the LEAP, the energy density is less than that in TEM and controlled destruction of the specimen (approaching one atom at a time) is the goal. In other words, specimen damage in the LEAP is not as significant an issue as in electron microscopy because the LEAP does not image the same specimen region for a long period of time in order to measure electron scattering. Thus, the LEAP approach can obtain atomic level resolution of organics that is superior to TEM biological resolution. In addition, TEM does not provide elemental identification of low atomic number elements, does not provide any elemental identification of single atoms, and does not provide true 3-D imaging, as does the LEAP.

For LEAP specimen preparation, biological structure is maintained by removing the specimen water and replacing it with a resin or polymer with a low vapor pressure, similar to that done for TEM or SEM to preserve structure in the vacuum. A secondary approach is to freeze the water in place and to maintain this frozen state during subsequent preparative steps and while imaging. A third approach is to stabilize structure by embedding the sample in non-organic materials.

Condition 2—stable in high electric field: This condition, as well as condition 3, relates to the special needs for atom probe specimens to be stable in an electric field on the order of 10 V/nm. Briefly, biological materials must possess sufficient internal bonding stability so that they do not unfold or lose conformation in this high electric field. In short, the biological sample cannot simply “fly off” as whole molecules or fragments before the threshold voltage to perform the analysis is attained. Imparting high-field stability to the sample can be achieved by embedding the material in a supporting matrix, as described for Condition 1. Additional stability can also be achieved, as required, by introducing covalent cross-links within the specimen by reaction with suitable reagents such as aldehydes, dialdehyde, osmium tetroxide, ruthenium tetroxide, benzophenones, and other cross-linking chemistries. In short, various cross-linking reagents will suffice. Other strong bonds, such as metallic and ionic bonds may also be used to stabilize the structure during analysis. With some specimen preparations, there may also be a need to introduce cross-links between the specimen and the supporting substrate.

Condition 3—Field ionization from specimen surface as atoms or small molecular ions: This condition also relates to the special needs for atom probe analysis of materials. In order to achieve ideal spatial resolution and molecular identification (within any given mass spectral resolution), it is necessary that individual specimen atoms or small molecular fragments should ionize, rather than large molecular clusters. Ionization must also be controllable such that it occurs only due to voltage pulses (or due to other ionizing radiation pulses), and it must only occur from the top atomic layer of the specimen. With respect to ion size, smaller is better to achieve high spatial resolution and facilitate elemental identification. Ideally such ions should be single atoms or small molecular fragments. It is less desirable, but still acceptable, for ions to be small molecular fragments (generally ten atoms or less, exclusive of hydrogen). It is not very desirable to ionize large fragments (generally greater than 10 atoms). Any ion larger than the spatial resolution of the LEAP will degrade spatial resolution. (Spatial resolution in

TABLE 1
Ionization probability in atom probes and types of bonds found in
different materials. The third column indicates the extent of
experience and types of materials with different bond types that
have been examined with atom probe microscopes.
In an Atom Probe: Ionization Probability α (Σ_{bond energies}, geometry)

| Material bonding | Bond energy (kcal/mol) | Atom probe analysis? |
|---|---|---|
| Metallic bonds | 27-83 | Extensive |
| Covalent bonds | 15-170 | Si, C (diamonds) |
| Ionic bonds | 140-250 | Rutile (TiO₂) |
| Hydrogen bonds | 1-12 | ? |
| van der Waals | 1-10 | ? |

Metals and microelectronics: metallic, covalent bonds
Biotechnology materials: covalent, ionic, dispersion, hydrogen bonds

the LEAP is nominally 0.5 nm laterally and 0.2 nm axially. However, this is also a function of the geometry of the specimen and other specimen and instrument factors.) The ion size may be controlled by specimen preparation and other means.

Briefly, the ionization voltage for a given specimen atom is proportional to the bond strength of that atom within the specimen, and may also be influenced by the specimen conductivity and geometry. The ion mass. or size (the number of atoms in a molecular cluster) may be controlled, by varying the bond strength within the sample. This is done by introducing strong bonds within the specimen and/or by varying the level of cross-linking within the sample and/or between the sample and an embedding matrix. The bonds are primarily covalent, but may also be ionic, or metallic as these are also high strength bonds. Weak bonds, such as hydrogen bonds and dispersion or dispersion bonds (van der Waals bonds) are generally insufficient unless there are multiple such bonds. Creating suitable bonds is achieved in practice by treating the specimen with a cross-linking agent, such as (for example and without limitation) glutaraldehyde and osmium tetraoxide, as well as other agents. In general, the more highly cross-linked a specimen is, the smaller the fragments that will be ionized because each atom or ionizable moiety will be more tightly bound to its neighbors. This will ensure that the bond(s) that will break upon field ionization will be the weakest bonds in the highest field zones. These will be the bonds closest to the local electrode in the LEAP, or those closest to the remote electrode in a conventional atom probe. Thus, the surface atoms will field evaporate preferentially as compared to atoms below the specimen surface.

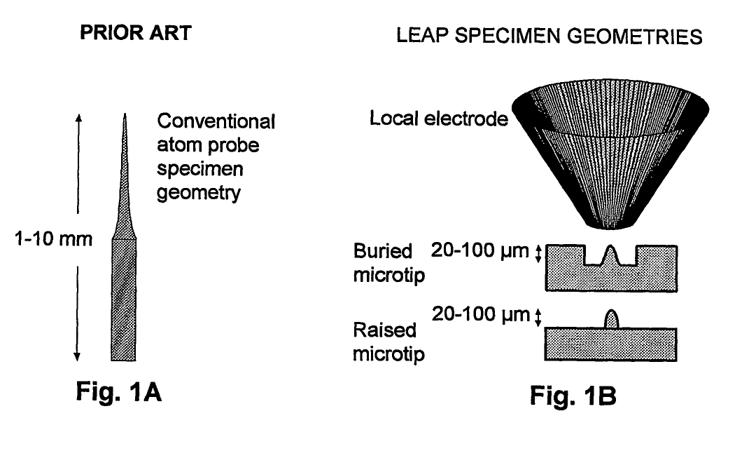

Condition 4—Needle-like (acicular) specimen geometry: The specimen must have a needle-like geometry in order to focus the high electric field of several V/nm at the specimen surface. With conventional atom probes the needle must be several millimeters long. In contrast, with the local electrode atom probes such as the LEAP, the needle may be much shorter, on the order of a few tens of microns. This is because in the LEAP approach, the local electrode applies the field only in the vicinity of the specimen tip (see FIG. 1B). With the local electrode, the needle only needs to be about as tall as the aperture of the local electrode. Few biological materials are sufficiently sturdy to maintain even a 10-100 μm-long needle shape with a circa 100 nm radius tip, especially after water is removed. However, replacing specimen water with an embedding material of sufficient physical strength can provide the necessary mechanical support.

Condition 5—Uniformly curved and smooth specimen tip: Imaging atom probes are projection microscopes where the specimen itself is the lens. Minimal aberration is achieved when the lens, that is the tip of the needle-shaped specimen, is a smooth hemispherical surface. Although at the atomic scale it is impossible for the surface to be entirely smooth because each atom has a finite size, roughness larger than the size of individual atoms will introduce field irregularities that will convolve and degrade the image resolution. To achieve a smooth surface, the entire specimen must uniformly ionize one atomic layer at a time. This, in turn, is realized by ensuring that there are no large variations in the bond strengths of the material that would cause weakly bound atoms (or small molecules) to field ionize from a location. that is not at the specimen surface. In practice, this is achieved by stabilizing the internal bonds of the sample, and by having an embedding or supporting matrix that field evaporates small ions within the same field strength range as the embedded sample that is to be analyzed.

Condition 6—Sufficient level of electrical conductivity: The specimen must have a sufficient level of electrical conductivity to concentrate the electric field at the specimen apex. This condition is required to conduct electrons from the tip of the specimen as atoms or small molecular fragments field evaporate as positive ions (as in the LEAP). For example, in one embodiment the LEAP uses nanosecond duration electric pulses to induce field evaporation of specimen ions. The specimen, therefore, must have a sufficiently low impedance to conduct this charge rapidly to and from the specimen tip. With longer duration pulses the need for low impedance specimen preparations correspondingly decreases. When the pulsed ionization field used to induce single atom or small molecular fragments to ionize from the specimen surface is non-electric (such as photonic), insulating specimens may be analyzed.

In summary, most of these necessary conditions may be achieved by removing the specimen water and replacing it with an embedding matrix that imparts stability in vacuum, stability in the electric field, preserves structure, enables ionization of small molecular fragments and/or single atoms, and

TABLE 3
Additional properties for embedding matrix materials.

Freely diffusible into specimens to ensure good infiltration
Minimal disturbance of structure during infiltration and polymerization
or cross-linking or other processes to support embedded specimens
Low out-gassing once in solid state, at least at cryogenic temperatures
generally used in atom probe microscopes.
Bond energy in same range as biological molecules. In most cases this is
achieved with mostly covalent and/or ionic bonds as are common in
organic materials.
Discernable atomic structure from proteins, lipids, and other biological
and organic materials that are to be encapsulated and examined with the
LEAP. Because matrices will often be organic in composition, different
embedding materials will be best suited for different specimens in order
to enable discrimination of the embedment from the specimen.
Uniform density and small domain size, on the order of a few nanometers,
in order to provide structural uniformity.
For a voltage pulsed atom probe using pulses of nanosecond duration the
material will require electrical conductivity greater than about
10⁻² ohm-cm. Any necessary conductivity must also occur with a
sufficient level of spatial uniformity within the matrix.
Specimens are generally imaged at cryogenic temperature during LEAP
analysis. For polymers this would be at least below the polymer glass
transition temperatures. Therefore, when conductivity is required the
embedding matrix must provide this at such temperatures, which will
typically be as low as 173 K (a very low polymer glass transition
temperature). Maintaining conductivity at even lower temperatures is
desirable.

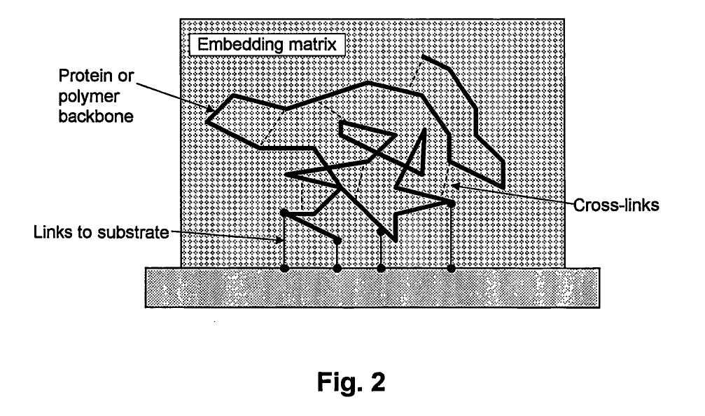

provides adequate electrical conductivity. These considerations are shown schematically in FIG. 2. As depicted in FIG. 2, a protein or polymer specimen is shown as a black ribbon. The specimen can be internally stabilized by introducing cross-links within the specimen molecule, as shown by broken lines in FIG. 2. The specimen can also be externally stabilized by introducing covalent links to the substrate, as shown the thin solid lines in FIG. 2. The entire specimen is embedded within a conductive embedding matrix, preferably an ICP. Additional ideal properties desired for embedding materials are presented in Table 3.

In addition to removing specimen water and replacing it with an embedding matrix, it is also possible to freeze the water in place. It is also possible to combine these approaches and freeze the water, while also embedding the specimen within a hydrogel or other polymer. This polymer or macromer matrix can enhance mechanical stability, potentially provide some electrical conductivity (depending upon the polymer or macromer and any staining or other treatment), and provide chemical bonds to the water and to the specimen to provide for uniform ionization of the embedded specimen and the embedding matrix. When the specimen and/or the embedding matrix includes frozen water, the atom probe must allow for cryogenic transfer so that a frozen specimen can be introduced into the vacuum and simultaneously be kept frozen until the analysis is complete.

(4) Specimen Preparation to Achieve Atom Probe Analysis of Biologicals and Organics:

Intrinsically conductive polymers (ICP) provide a nearly ideal combination of properties for embedding biological materials; namely, adequate electrical conductivity and organic polymer composition. Moreover, ICPs have intramolecular organic bonds that are similar (or identical) to those found in biological specimens. The ability to atom probe an ICP has been reported for polypyrrole (Maruyama, Hasegawa et al. 1987).

Several ICPs have properties that make them useful for embedding atom probe specimens. These include polyaniline, with a reported conductivity as high as 10 S/cm (e.g., the commercially available “Panipol F” and “Panipol T” brand resins, from Panipol Ltd, Porvoo, Finland) and polythiophenes, with reported conductivities as high as 30 S/cm. Several polythiophene formulations are available from H.C. Starck (Newton, Mass., Germany, and other locations) including “Baytron M” and “Baytron P.” Additional formulations are produced by TDA Research of Wheat Ridge, Colorado, including Oligotron™ and Aedotron™ materials.

These ICPs have physical properties that make them adaptable for embedding biological tissues, proteins, cells, macromolecular complexes, biotechnology materials and devices, particulates, porous materials, and the like. The desirable properties of ICPs as embedding matrices include:

Low viscosity, especially as monomers.

Reasonable viscosity as prepolymers and polymers.

The Panipol-branded and Baytron-branded resins can be blended with other resins and cross-linkers to alter or to optimize their embedding properties. (Sufficient conductivity to perform LEAP is maintained in blends containing these polymers.)

Baytron-brand polythiophene is suitable for freeze substitution methods of specimen preparation because it can be polymerized from monomeric precursors; 3,4-ethylenedioxythiophene and Fe(III) toluenesulfonate (“Baytron M+C”-brand precursors). The polymerization takes place at low temperatures within organic solvents (such as toluene and alcohols) that are routinely used for TEM specimen preparation. This material also has suitable properties for non-cryogenic embedding protocols. The recently developed TDA polythiophene block copolymers sold under the Oligotron™ trademark are also useful for various methods of embedding. These polythiophene block copolymers have a variety of end groups, including poly(ethylene glycol) (PEG), poly(propylene glycol) (PPG), polydimethylsiloxane (PDMS), and acrylic derivatives such as the Aedotron™-brand formulation. These terminal groups impart different solubilities, including solubility in organic solvents, as well as provide different polymerization conditions and ultimate physical properties.

Aqueous dispersions of Panipol-branded and Baytron-branded ICPs have reasonably low viscosity. Thus, many biological materials may be embedded directly from water, with no initial dehydration step. The specimen is simply added to the dispersion and the dispersion is allowed to gel (or polymerize if monomers are used). The specimen is thereby entrained within the polymer network. This is a novel property for an embedding matrix. Direct aqueous embedding is not used in TEM. This type of embedding greatly simplifies protocols and provides adequate structural preservation for many imaging applications, including LEAP. There are additional ICPs that also have suitable properties (or which can be formulated to have such properties) as listed in Tables 1 and 2.

These embedment materials can also be used for embedding non-biological materials for atom probe analysis. These include certain types of polymeric devices, drugs, liposomes, organic-inorganic composites, nano—biotechnological devices such as bio-array chips, biosensors, biomaterials, and bio-microelectromechanical devices (bioMEMs). These materials can also embed a vast range of different types of nanoparticles and fragments of structures that require one or more of: mechanical support, void filling, a uniform ionization surface, electrical conductivity, and other properties (all of which can be provided by the matrix). Some additional materials that can benefit from embedding include fullerenes, ceramics, quantum dots, dielectrics, nano—and micro-porous catalysts, zeolites, materials with nanoscale voids and cavities, and other types of micro- and nano-particulate materials not explicitly listed herein.

These ICP properties enable biological and organic specimens to be prepared using protocols that are somewhat similar to those used for the preparation of TEM specimens to preserve specimen structure. Certain of these formulations will enable embedding from organic solvents, embedding from water or water-miscible solvents, and may enable high-resolution cryogenic immuno-cytochemical protocols using freeze substitution (Table 3).

In addition to ICPs, polymers that can be made conductive via extrinsic treatment may also be used to support and embed specimens and fill voids. Such materials may have a significant fraction of double bonds or other moieties that can be subsequently reacted with OsO₄ or RuO₄ to induce sufficient conductivity. Such materials include low-molecular weight dienes with hydroxyl groups and other water-soluble moieties, polymerizable diene monomers, unsaturated acrylics and epoxies, and other polymers or prepolymers that can be polymerized by free radical activation, photo-activation, or other means. These may then be cross-linked by OsO₄ or RuO₄ to provide further stability and some electrical conductivity. Of course, when an atom probe that does not rely on nanosecond pulsing to achieve ionization is used, then even lower conductivity polymers may be used to embed and stabilize biological specimens.

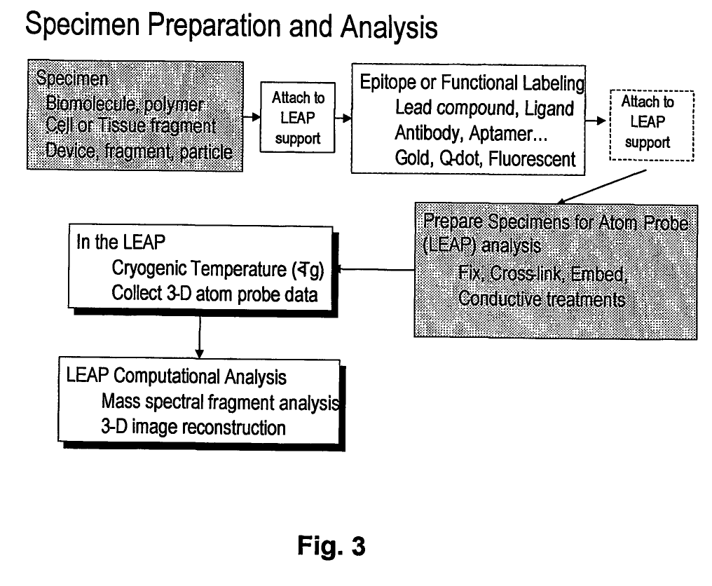

The overall approach to specimen preparation and LEAP imaging is diagrammed in FIG. 3.

TABLE 3
Intrinsically conductive polymers (ICP) for
embedding biological or organic specimens.

| | | | | Somewhat |
|---|---|---|---|---|
| | | | | like known |
| | | | | TEM |
| Protocols | ICP Name | ICP Chemistry | Solvents | resins |
| Organic | Panipol F | Polyaniline | Toluene | Spurrs, |
| solvent | Panipol T | solution | Toluene | Epon, |
| embedding | Baytron M + C | Polyaniline | Alcohols | Other |
| En bloc and | | solution | Acetone | epoxies, |
| on tips | | Polythiophene | | Some |
| | | monomers* | | acrylics |
| Aqueous | Panipol W | Polythiophene | Water | Lowicryl |
| embedding | Baytron P | dispersion | | K4M, |
| En bloc and | Baytron | Polythiophene | | LR White |
| on tips | CPUD2 | dispersion | | |
| | | Polyaniline | | |
| | | dispersion | | |
| | | blend | | |
| Freeze- | Baytron M + C | Polythiophene | Alcohols | Lowicryl |
| substitution | | monomers* | Acetone | HM20 |
| En bloc and | | | | |
| on tips | | | | |
| *The polythiophene monomers are 3,4-ethylenedioxythiophene and Fe(III) toluenesulfonate or other dopants. Panipol is available from Panipol Ltd. (Porvoo, Finland), Baytrons from H. C. Starck, Inc. (a wholly-owned subsidiary of Bayer Corporation, Leverkusen, Germany, and Newton, Massachusetts), and | | | | |
| # polypyrrole from Sigma-Aldrich Chemical (St. Louis, Missouri). A new family of polythiophene block copolymers is produced by TDI (Wheat Ridge, Colorado) with different block components that alter solubility and other material properties that will make these potentially useful for organic, aqueous, and cryogenic protocols. | | | | |

(5) Increasing the Conductivity of ICP Embedding Materials:

Because OsO₄ fixation provides strong cross-links to stabilize specimens, and can provide some conductivity and staining of specimens, the effects of OsO₄ treatment on the resistance of “Baytron CPUD2”-brand polythiophene was examined. Interestingly, the resistance of cast Baytron CPUD2 ICP (two-point measure) decreased from 10- to 50-fold upon a 10- to 60-minute exposure to dry saturated OsO₄ vapor in a closed vial. Cast “Baytron P”-brand polythiophene also decreased its resistance to about ¼ of its initial value in a similar time frame. The decreased resistance is found throughout the bulk material. This was confirmed by removing the exposed surface of the various Baytron-branded polymers and measuring resistances again. The decreased resistance was found througout the bulk material, and thus was not a function of a surface coating of osmium. This was further confirmed by examining cross sections of the OsO₄-stained resins with high-resolution secondary and back-scattered SEM. These observations indicated that the OsO₄ had penetrated into the bulk of the material. Hence this was not a surface deposition effect. This demonstrates that osmium-treated polythiophenes have good conductivity properties for embedding biological and other materials. This also demonstrates a method to increase (quite significantly) the electrical conductivity of polythiophenes that may be useful for other applications of conductive polymers.

Because atom probe analysis is routinely performed at cryogenic temperatures, it is critical to determine how such temperatures affect electrical conductivity. The resistivity of untreated Baytron P and CPUD2 specimens were assessed by continuous monitoring of DC resistance as the temperature was systematically decreased in a cryostat. It was observed that CPUD2-brand polymer increased its resistivity no more than two-fold (doubled) as the temperature was decreased from 298K to 97K Baytron P-branded polymer increased its resistivity also by about 2-fold over the same temperature range. 97K is well below the glass transition temperatures (T_{g}) for polymers and organics. Typical T_{g}'s for these materials are no lower than 120-170K Such a doubling of resistance is minimal and thus does not have an adverse effect on the use of these materials in embedment formulations and where specimens are examined at cryogenic temperatures.

(6) Specimen Preparation Benefits of Osmium or Ruthenium Treatment:

Treatment with osmium or ruthenium also improved the physical properties of Baytron P- and CPUD2-branded polymers, as well as PANI F-branded polymer, when preparing atom probe specimens. These materials were made substantially more rigid and tougher, and thus were more easily cut to the proper shape and were more readily sharpened with an ion beam into the proper needle-shape geometry. An additional benefit of OsO₄ treatment is that osmium provides a high mass element for mass/charge calibration. While simple OsO₄ vapor exposure was used to treat these materials, additional methods can also be used, including treatment in aqueous or solvent solutions, treatment at cryogenic temperatures (within solvents), and a recently developed method that uses low voltage discharge plasma chemical vapor deposition of OsO₄ (Akahori, Handa et al. 2000). Such methods can be applied as required to enhance conductivity and to stabilize specimens prior to embedding within an ICP (or other polymer), as well as after embedding within an ICP (or other polymer).

(7) Additional Conductive Stains and Cross-Linkers:

Many methods have been developed to increase the.conductivity of biological specimens for TEM. These include metallic “negative” stains such as uranyl and lead salts, reaction of membranes and synthetic polymers with OsO₄ or RuO₄, and sequential protocols with OsO₄ and thiocarbohydrazide. The latter-most method, known as the OTO method, provides sufficient conductivity to examine complex cellular structures with SEM (Allen, Jack et al. 1986; Goldstein, Newbury et al. 1992; Bozzola and Russel 1999). In conjunction with cryogenic and non-cryogenic methods, OsO₄ treatment also yields hydrated polymers that can be imaged by TEM and SEM (Goodman, Li et al. 1988; Li, Goodman et al. 1988; Goodman, Simmons et al. 1990). These sample preparation methods can also be applied to atom probe analysis. As with the OsO₄ treatment, the heavy metals can also be used to facilitate mass calibration.

(8) Method to Prepare Materials into the Suitable Geometry:

To use the above-described materials for embedding and stabilizing biological materials it is necessary to form or shape them into the proper geometry. This may be achieved in several ways, each of which will be more suitable for a given type of specimen.

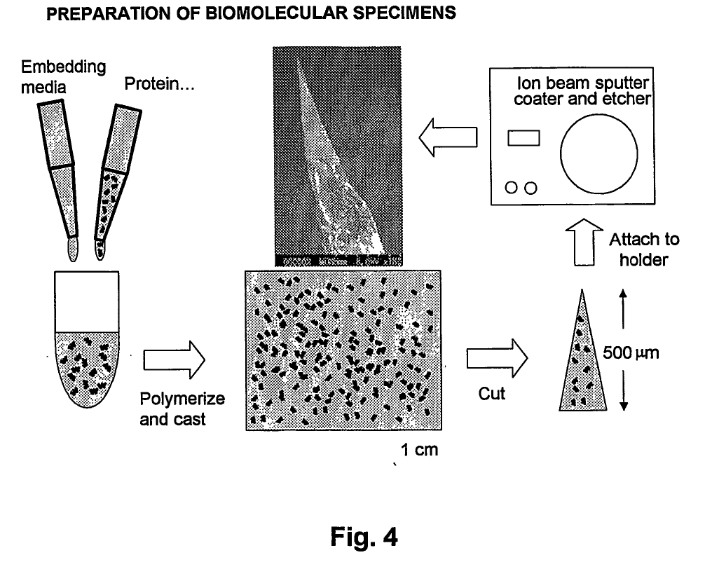

Preparing specimens from cast blocks (en bloc methods) is quite useful for many specimens, but requires subsequent processing to obtain the necessary configuration for analysis. FIG. 4 shows the overall protocol and an SEM micrograph of a atom probe specimen tip prepared using this method. As shown in FIG. 4, the embedding matrix and the specimen to be imaged (a protein in the case of FIG. 4) are mixed together and the polymer allowed to gel (or the monomer allowed to polymerize). The specimen is thereby embedded within the matrix. The en bloc embedded specimen is then cut into a micron-scale sharp tip, and attached to a suitable holder for LEAP or conventional atom probe analysis. The specimen is then fine sharpened with a broad argon ion beam (BIB) or with energetic gallium ions in a focused ion beam (FIB) instrument.

The particular specimen shown in the micrograph in FIG. 4 is an ICP embedding media that does not contain any protein, but does contain a polyurethane. Similar specimens have also been prepared with encapsulated proteins. The specimens may be cut as shown by several ways. These include careful trimming by hand with the aid of a very sharp fine point scalpel and a dissecting microscope, by thin section microtomy, and with a micromanipulator apparatus equipped with fine knives specifically designed for this purpose (not shown).

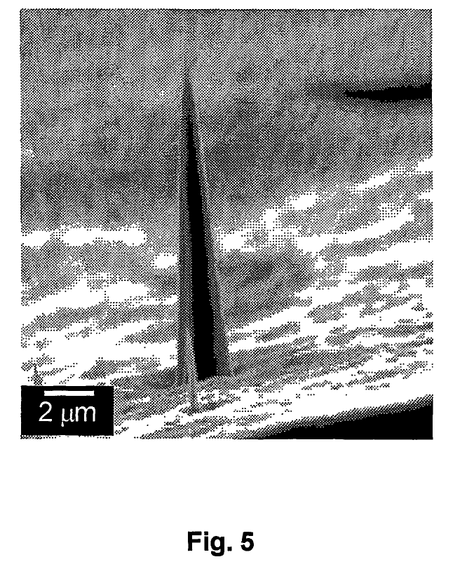

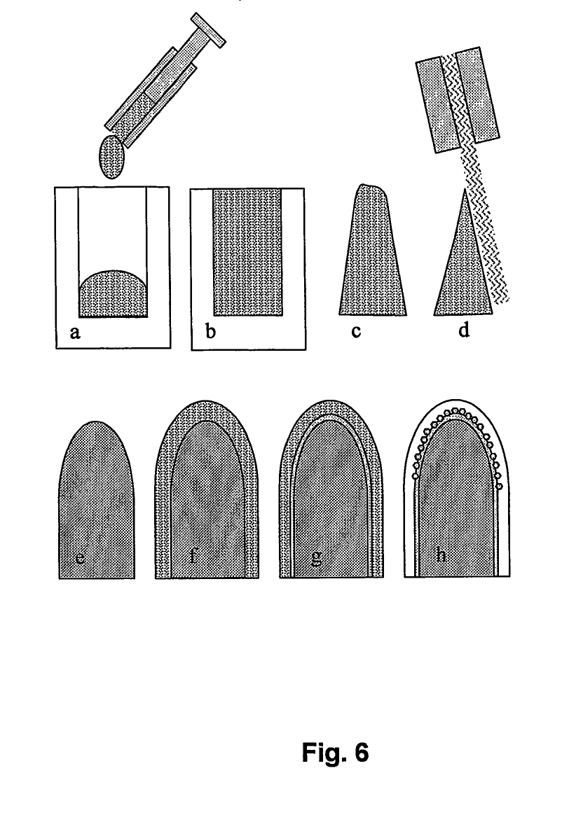

The SEM micrograph in FIG. 4 shows the tip of an osmium-treated Baytron CPUD2-brand ICP that has been mounted on the end of stainless steel needle. Such specimens may be imaged in the LEAP and in conventional (non-local electrode) atom probes and in field ion microscopes. The LEAP, as discussed, allows for the use of much shorter needles. These may be prepared from ICP embedding materials using broad ion beam (BIB) milling or etching. An example of such a microtip is shown in FIG. 5. Further discussion of this method of specimen preparation follows. Additional methods to prepare specimens are presented in FIGS. 6 to 11.

Specimens embedded in-an ICP matrix may also be coated onto pre-sharpened tips prepared from metals or conductive silicon (see FIGS. 6e and 6f). As required, any additional sharpening may be readily performed with a focused ion beam (FIB). Wetting agents are used, where required, to enhance close adhesion and flow onto the surface or into interstices in the substrate material surface. Suitable wetting agents for Baytron-brand ICPs include isopropranol and ethylene glycol. To ensure tight adhesion to presharpened tips or planar surfaces, adhesion agents such as silanes (e.g., tetraethoxysilane) that strongly bond to a variety of materials, and thiols that bond to gold and some other metals, are used to improve the bonding stability between the ICP matrix and the substrate material. Adhesion-promoting agents may be mixed into the ICP, or tips may be pre-coated with a suitable adhesion promoting chemistry (see FIG. 6g ). The protein or other specimen may also be covalently attached to the tip prior to encapsulation within the ICP (see FIG. 6h ).

(9) ICPs as En Bloc Embedment Matrices:

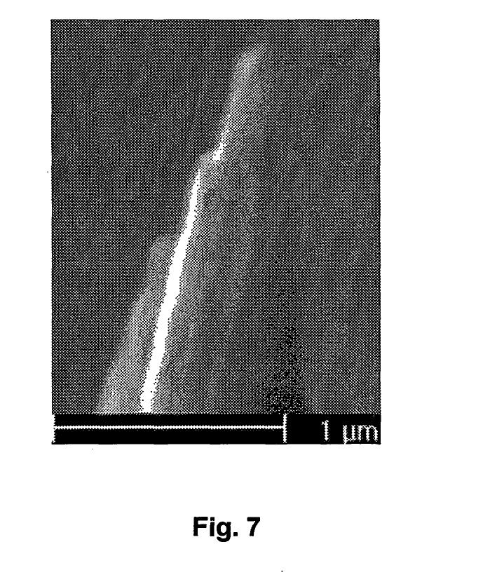

ICPs may be prepared as monolithic or solid specimens. Monolithic specimens are made by casting or polymerizing specimens within blocks of ICP. For LEAP analysis, the specimen embedded into the ICP matrix is then cut into the proper tip geometry, as illustrated schematically in FIGS. 6a to 6d. This is analogous to en bloc embedding for TEM, where the specimen is first embedded and then is subsequently sectioned for TEM imaging. Many separate specimens can be prepared from a single block. The feasibility of this approach has been demonstrated by casting Baytron CPUD2-brand polymer, rough shaping with a scalpel, and final sharpening using an ion beam into an approximately 150 nm diameter tip. See FIG. 7, which is a SEM micrograph of the resultant tip.

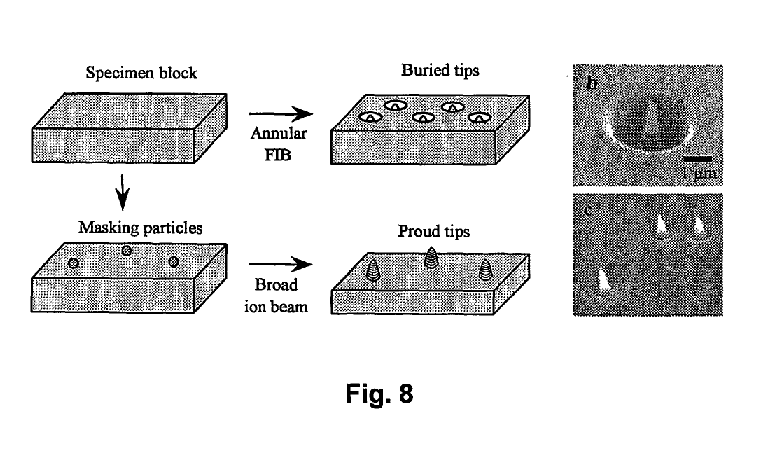

The present invention also includes additional methods to prepare multiple tips on a single specimen block, thereby facilitating high-throughput LEAP analysis (see FIG. 8). In one method, illustrated in the top panels of FIG. 8, a focused ion beam instrument (FIB) is used to carve out an annulus to create a buried tip at any desired location on the block (reference “b” in FIG. 8). In the second method, masking particles such as diamond dust or metal colloids are placed on the specimen block. The block is then irradiated uniformly with energetic ions at normal incidence to sputter (erode) the surface (Larson, Wissman et al. 2001). At the moment when the masking particles are sputtered to zero mass, multiple tips are left behind that stand clear of the flat surface (see reference “c” in FIG. 8). This method can be used to create dozens of tips on a single specimen block, simultaneously. This method is also quite useful for making multiple tips on a specimen in applications where it is not critical which regions are analyzed. If the masking. particles are carefully placed on areas of interest, then specific specimen regions can be selected for LEAP analysis. Such tips are shown in the micrographs in FIG. 6 and FIG. 8.

(10) Covalent Attachment of Specimens to Tips:

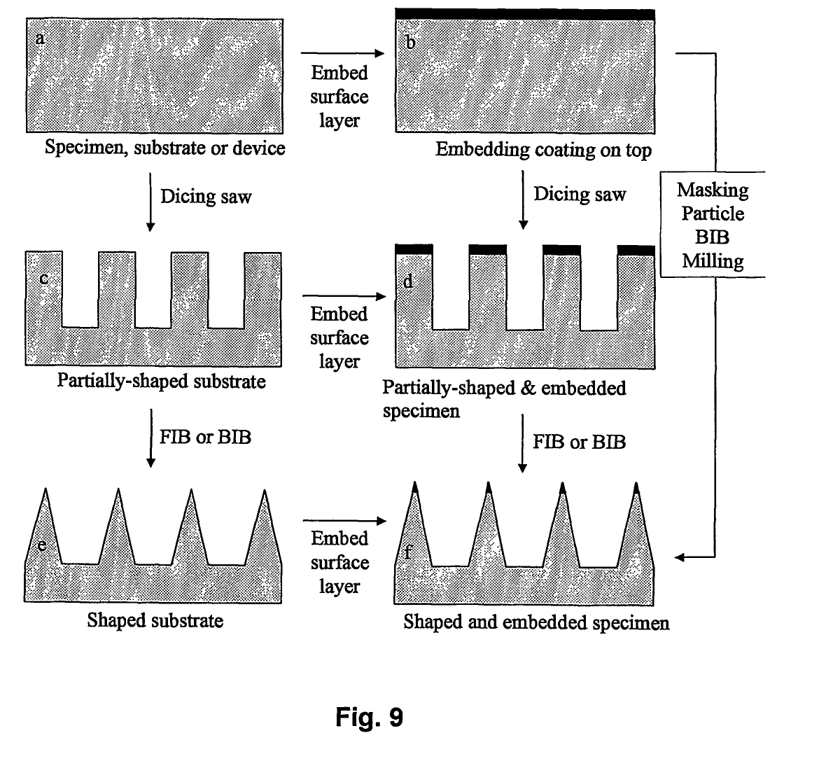

Biological and organic specimens such as proteins should be strongly attached to specimen supports to ensure that they ionize in good order (see FIGS. 6g, 6h, and the flowchart in FIG. 9). Self-assembled monolayers (SAMS) of organosilanes and thiols are widely used for this purpose in a variety of biotechnology devices with minimal effect on protein function, and by inference, protein structure. SAMS based upon thiols provide very flexible chemistries to link proteins (and ICPs) to gold surfaces. For example, SAMS may be used to link to a variety of functional groups and can be used to pattern protein adsorption (Prime and Whitesides 1991; Mrksich and Whitesides 1996; Ulman 1998). Amino terminal gold-thiol SAMS have been used to link DNA to gold tips for FIM imaging (not shown). Similarly, organosilanes that covalently bond to silicon (and other materials) are available with many different functional groups to bond to biological specimens (Heiney, Gruneberg et al. 2000; Kumar, Maldarelli et al. 2001) and to adhere to a variety of surfaces including metals and silicon. As shown in FIG. 6g, a SAM can be used to enhance bonding of a specimen-containing matrix. Or, as shown in FIG. 6g, the specimen can be bound to the needle via a SAM, and then the needle coated with an embedding matrix.

(11) Coating ICP Embedments on Surfaces:

Coatings of ICP embedments can be applied to the surfaces of devices and materials to preserve biologicals or other materials on that surface for LEAP analysis. Coatings of ICP embedments on surfaces can also be used as a method of preparation of non-adherent study objects, such as a protein or a nanoparticle. In this case, the study object is mixed with the ICP matrix and is then applied to a planar surface or to a partially formed atom probe specimen. This method has been demonstrated using a Baytron-brand formulation (called CPG 130.6) that contains tetraethoxysilane as a linking agent to enhance bonding to a silicon substrate.

There are several ways to prepare substrates into suitable specimens for LEAP analysis as diagrammed in FIG. 9. One step is to use a dicing saw or other cutting tool to produce regular posts that are then further sharpened with a focused ion beam (FIB) or a broad ion beam (BIB). Specifically, as illustrated in FIG. 9, a bulk specimen to be examined or a substrate are designated at reference (a). If the specimen itself is amenable to shaping, the specimen can be shaped using a dicing saw or by photolithography, or by any other means now known or developed in the future for shaping substrates. The rough-hewn specimen is shown at reference (c). The rough-hewn specimen can then be finished into a plurality of needles standing clear of the substrate using FIB machining or by any other suitable means, as shown in reference (e) of FIG. 9.

Alternatively, if the neat specimen is not amenable to shaping, the specimen can be embedded in an matrix to yield an embedded specimen. The embedded specimen is than disposed onto or otherwise immobilized onto the substrate, as shown at reference (b) of FIG. 9. The coated substrate is then machined as described in the previous paragraph and as illustrated in panels (d) and (f) of FIG. 9.

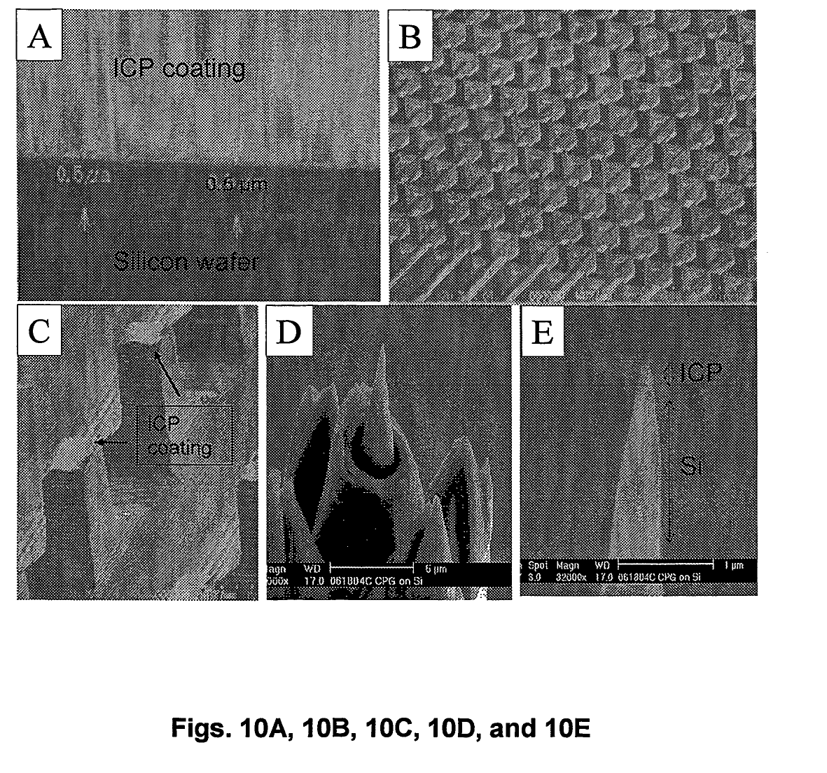

Scanning electron micrographs that illustrate the process depicted schematically in FIG. 9 are shown in FIGS. 10A to 10E. In FIGS. 10A-10E, a silicon wafer, as used in microelectronics and many types of biotechnology devices, was used as the substrate. A mixture of Baytron P with11.9% by weight tetraethoxysilane (designated as CPG 130.6 by the manufacturer, H.C. Starck) was applied to silicon wafers using spin casting, followed by a two-step curing at 50° C. for 10 minutes and 180° C. for 180 minutes. By careful control of the spin casting speed, a uniform coating of about 0.5 μm can be achieved. A photomicrograph of the.coated substrate is shown in FIG. 10A. The figures are Scanning Electron Microscopy (SEM) images illustrating the uniformity of the CPG ICP coating (FIG. 10A). The coated substrate was then diced, with the results shown in FIG. 10B. The ICP coating is present following dicing, as is clearly illustrated in FIGS. 10B and 10C, and is still tightly adherent following FIB sharpening. as shown in FIGS. 10D and 10E. Prior to FIB sharpening (i.e., after the step illustrated in FIGS. 10B and 10C but before the step shown in FIG. 10D), the ICP-coated substrates were exposed to saturated OsO₄ vapor for 1.5 to 2 hrs in a sealed container.

The specimen comprising a sharpened ICP needle on a silicon post may be examined in the LEAP by placing the local electrode directly over the tip (as diagrammed in FIG. 1b). Alternatively, the silicon post may be removed from the wafer and attached to a metal needle or similar other support for analysis with a conventional atom probe (or with the LEAP). The methods illustrated in FIG. 9 and in FIGS. 10A through 10E can also be combined with the inventions disclosed in patents U.S. Pat. Nos. 6,576,900 and 6,700,121 (which disclose pre-formed and partially pre-formed atom probe specimens to enable rapid preparation of LEAP specimens).

(12) Cryogenic and Non-Cryogenic Preparation:

Cryogenic preparation generally can provide excellent preservation of many specimens, as demonstrated with isolated proteins, proteins within membranes, macromolecular complexes, viruses, and other biological structures (Walther, Chen et al. 1992; Colliver, Brummel et al. 1997; Baker, Olson et al. 1999; Bozzola and Russel 1999; Auer 2000; Baumeister and Steven 2000). Because frozen hydrated specimens have minimal electrical conductivity, it is necessary to introduce conductivity within the specimen during a freeze substitution step when nanosecond electrical pulsed LEAP is used for analysis. Alternately, all specimen water may be removed during freeze substitution. This is achieved by rapidly freezing (˜10⁴ or 10⁵° C./second) so that water forms amorphous or vitreous ice. This is most commonly done by plunging the specimen into a cryogen such as liquid ethane cooled by liquid nitrogen, or by other methods such as high-pressure, slam, and propane jet freezing. Specimens are then cryogenically transferred to organic solvents such as ethanol or acetone, fixed, and then embedded at cryogenic temperatures. The specimen may then be prepared at room temperature, or they may be cryogenically sectioned and immuno-labeled or stained at reduced temperatures. Freeze substitution is generally preferred for immuno-histochemistry because it preserves antigenic conformations and enzyme activity (Beesley 1989; Jayasena 1999; White, Sullenger et al. 2000; Shiurba 2001). These functional indicators are strongly suggestive that near-native biological structure is maintained. As discussed above, the solvent-based ICP materials are suitable for freeze substitution.

All protocols require care in order to maintain the highest level of structural preservation. For example, many procedures will require careful dehydration through sieve-dried ethanol for organic solvent-based ICP embedments (e.g., Panipol F, T and Baytron M+C), and drying from low surface tension solvents (e.g. hexamethyidisilazane or HMDS), use of the critical point method, or freeze-drying when preparing materials from aqueous reagents (Albrecht, Rasmussen et al. 1976; Goodman 1999) will be necessary to preserve structure. Direct embedding with aqueous ICPs also provides a rapid protocol to achieve excellent preservation of hydrated structures.

(13) Specimen Preparation Pathways:

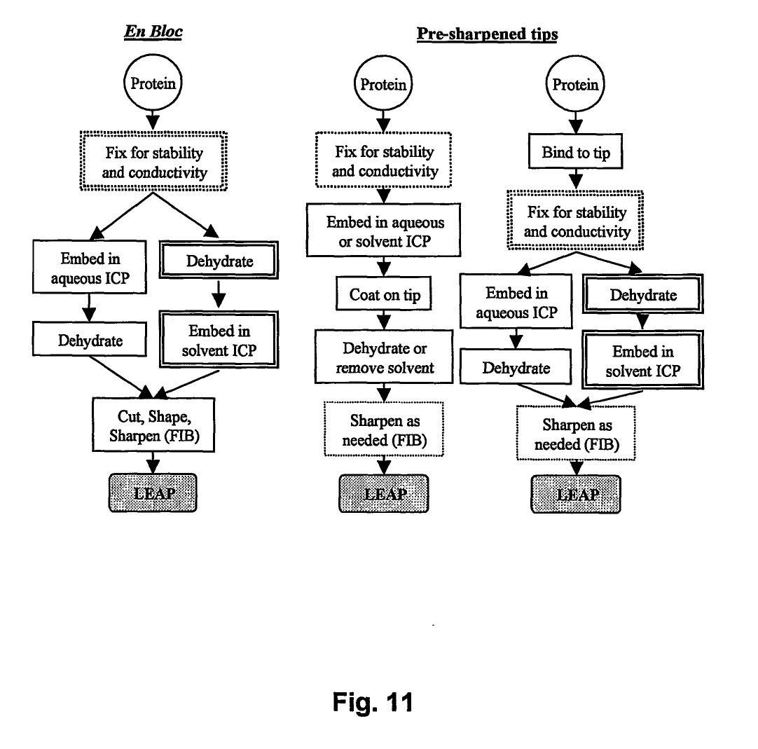

FIG. 11 diagrams several protocols for specimen preparation. Protein or other macromolecule- or nanoparticle-containing specimens may be encapsulated in a block (en bloc method), directly coated onto tips from a blend of specimen in ICP, or pre-attached to a support tip using linkage chemistries and then coated with ICP. Various chemical agent pre-treatments can improve the conformational stability of proteins, such as adding internal cross-links. Treating with osmium tetraoxide, ruthenium tetraoxide, uranyl acetate, glutaraldehyde, tannic acid, and/or potassium permanganate will increase conductivity. These treatments may be performed in solution (using centrifugation or filtration rinse steps), or after the protein is attached to the tip surface. These treatments may also be used to treat bulk specimens such as tissues or cells in culture. The protein or other material is then embedded into the ICP using aqueous prepolymers, solvent-castable prepolymers, or monomers. These methods can also be applied to non-biological organic and inorganic materials as required.

(14) Atom Probe Analysis of Organic Materials:

As demonstrated above, the present inventors have developed and adapted materials with suitable properties to be used as embedments for atom probe analysis of biologicals, organics, particulates and other materials as discussed. These materials have a uniform density as observed in SEM analysis. They can be shaped into the necessary acicular geometry. They have sufficient electrical conductivity for pulsed electric field LEAP analysis, and other desirable and necessary properties as outlined in Tables 1 and 2.

It is also necessary to demonstrate that it is possible to examine organic materials within the atom probe and obtain mass spectra consisting of small or low molecular weight ions, and to be able to map these ions to specimen locations to create meaningful and useful 3-D images of the material structure. The following results demonstrate the practical utility and operability of the present invention.

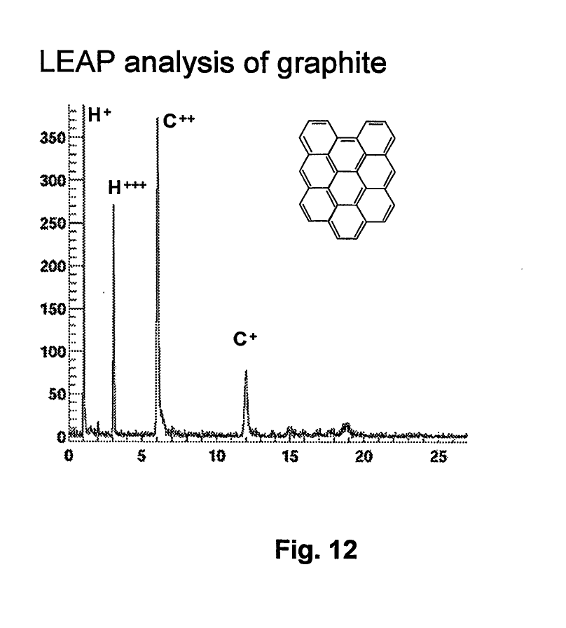

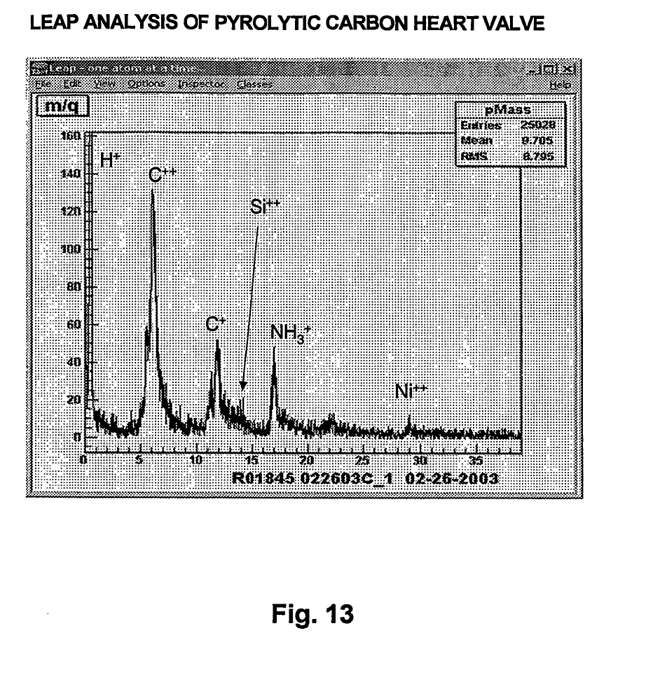

Meaningful mass spectra were obtained with the present invention for two model organic compounds: graphite (FIG. 12) and pyrolytic carbon (FIG. 13). It is very important to note that these straightforward, but highly cross-linked organic compounds ionized as individual atoms with a very small fraction of low molecular weight (MW) molecular clusters.

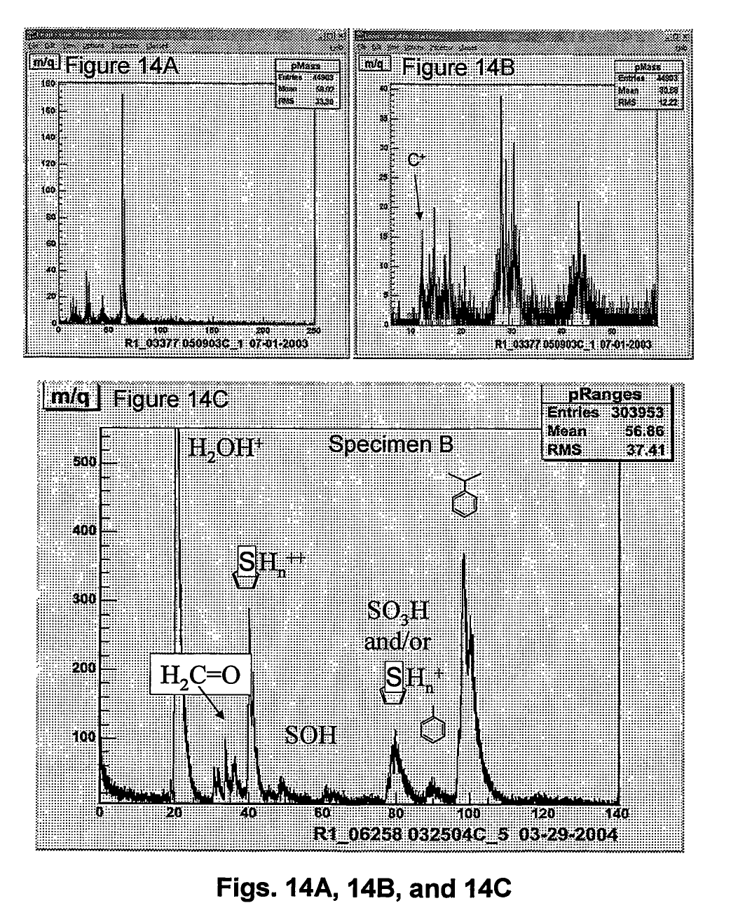

FIGS. 14A, 14B, and 14C demonstrate that osmium-treated CPUD2-brand resin can be imaged with the LEAP. (To the inventors' knowledge, these figures are the first atom probe images of an organic material, or of a polymer.) Furthermore, the ions of this material are of low mass/charge, and thus also of low molecular weight. FIGS. 14A and 14B are two different views of the mass spectrum of one sample of this material, while FIG. 14C shows the spectrum from an additional specimen with several tentative peak assignments. FIG. 14B is an enlarged segment of the full-width spectrum shown in FIG. 14A. Because the field strengths used to generate these spectra were low in relation to the field. strength required to ionize organic bonds, it can be assumed that the ions are in the singly-charged state. Thus, essentially all of the ions have a molecular weight of less than about 100 Daltons, with a significant proportion having a molecular weight less than 50 Daltons. As discussed above, this is highly desirable because it enables high spatial resolution and facilitates elemental compositional identification. It should also be noted that previous 1-D (compositional but not imaging) atom probe analyses of conductive polymers have reported much higher ion masses that are consistent with whole monomers, dimers, and trimers (Maruyama, Hasegawa et al. 1987). The prior art result is certainly not desirable for achieving good spatial resolution. The present improved result indicates that the sample preparation methods described herein, induding preparation of bulk specimens and osmium tetraoxide treatment, promote small-mass ions. In addition, the LEAP microscope may have also provided improvements in performance and localization of the ionization field compared to the instrumentation used in the earlier Maruyama et al. study.

While FIGS. 14A, 14B, and 14C show the mass spectra, it is difficult to assign elemental composition to each peak due to limited mass resolution with the particular LEAP instrument used for this analysis. This is because many different elements or combinations of elements within a molecular fragment can have quite similar masses that are not fully resolvable. Secondly, the ionization behavior of organic materials within the LEAP is not yet well characterized. It is anticipated that further study with a higher mass resolution LEAP device, in conjunction with the analysis of different materials of known composition, will facilitate the assignment of elemental and molecular composition to different observed mass/charge ratios.

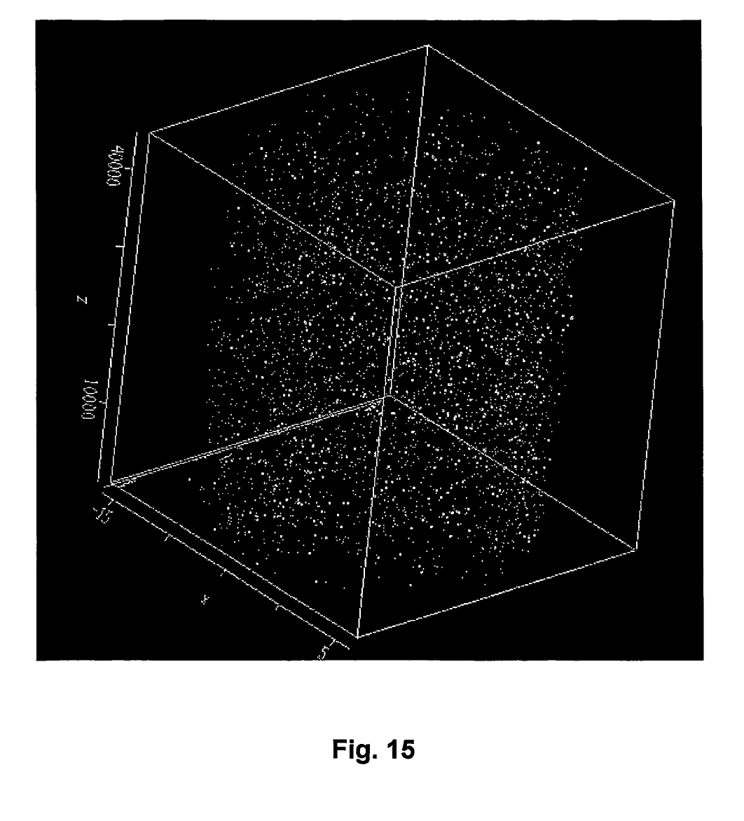

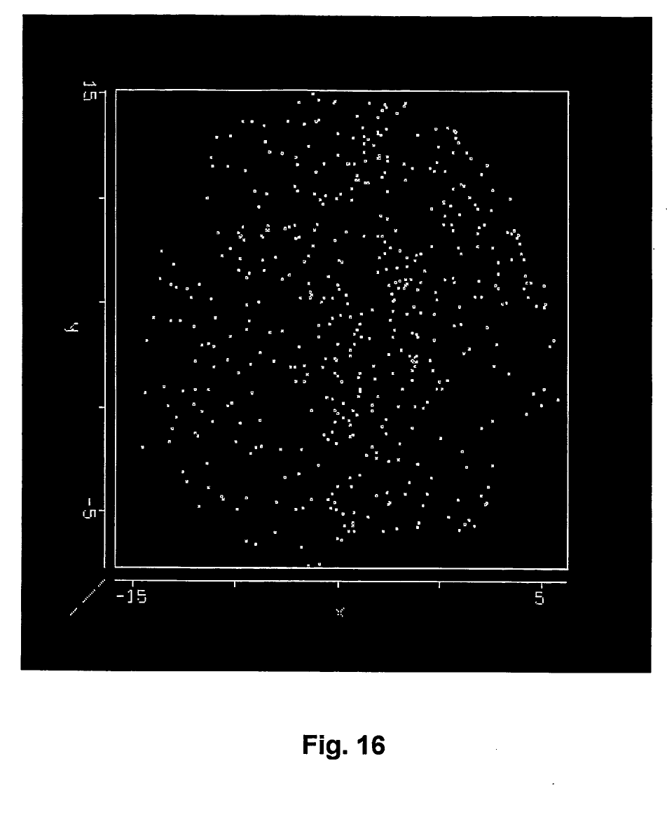

FIG. 15 is a view of a 3-D reconstruction of the ionized fragments of the same specimen whose mass spectrum is shown in FIGS. 14A and 14B. FIGS. 15 and 16 (which is a cross-section view of FIG. 15) illustrate that each mass fragment can be localized to a different 3-D location and thus indicate that reconstruction of the 3-D structure is feasible. The CPUD2-brand polymer contains three major polymeric components: a poly(thiophene), a poly(styrene sulfonate), and a poly(urethane). The material also contains other minor components. Although firm identification of the various ions has not yet been made, the 3-D reconstructed ion image of FIG. 15 shows that likely ions from each of these components are in separate areas, hence supporting the ability of the LEAP to image these structures. In addition, ion masses that should be co-localized if their identity is correct, such as the phenyl and sulfonate group constituents of poly(styrene sulfonate), appear in close proximity in the cross-sectional view of FIG. 16.

Finally, because polyurethanes have substantial chemical similarity to proteins, this indicates that these materials and the processes described herein can be used to enable the 3-D composition and structural analysis of proteins with the LEAP microscope.

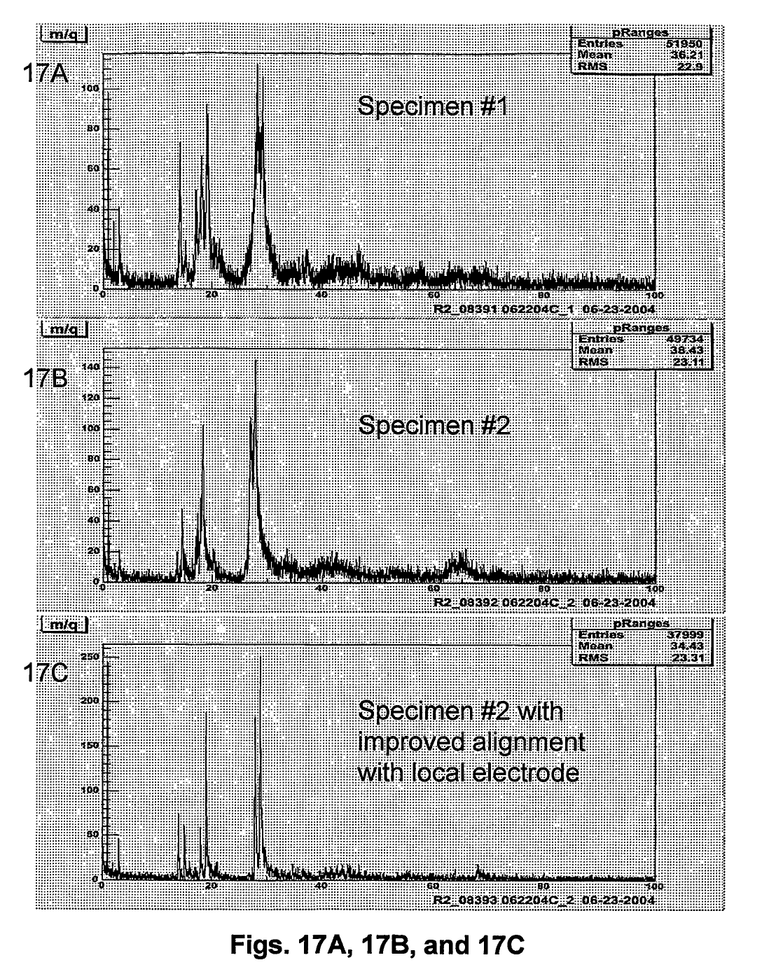

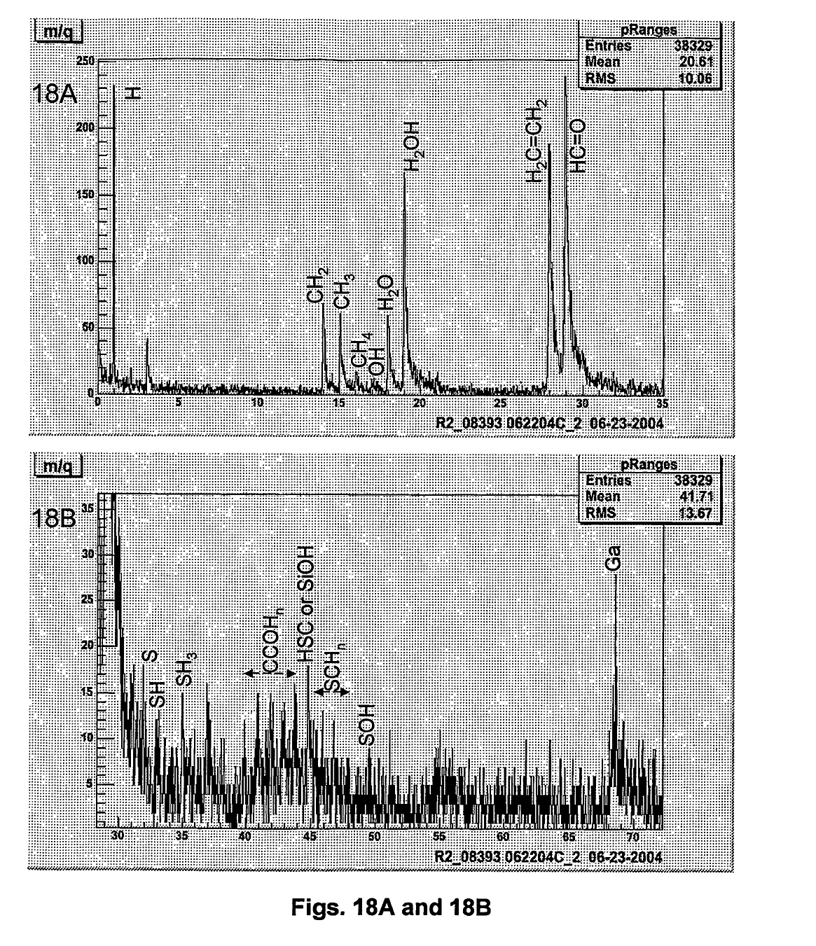

A second polythiophene formulation, known as Baytron CPG130.6 was also prepared for LEAP analysis by coating onto a silicon substrate as diagrammed and shown in FIG. 9 and FIGS. 10A through 10E. Unlike the CPUD2 formulation, the CPG 130.6 formulation does not contain polyurethane but does contain tetraethoxysilane to enhance bonding adhesion to silicon. Specimens of this material were examined with the LEAP and yielded the mass spectra shown in FIGS. 17A, 17B, and 17C. These spectra show that multiple identical specimens have the same spectral shape, thus demonstrating the repeatability of the process. These spectra are of somewhat higher mass resolution.than those shown in FIGS. 14A-14C. FIGS. 18A and 18B expand the mass spectrum shown in FIG. 17C into two ranges in order to identify the peak locations more clearly. FIG. 18A spans the atomic mass ranging from 0 to 35; FIG. 18B spans atomic mass ranging from about 30 to about 75. Several peaks are tentatively identified. Note that all organic components are of low masses and that multiple peaks are clustered by one atomic mass unit (AMU) separations. The Gallium peak is from implantation of some Ga due to FIB milling.

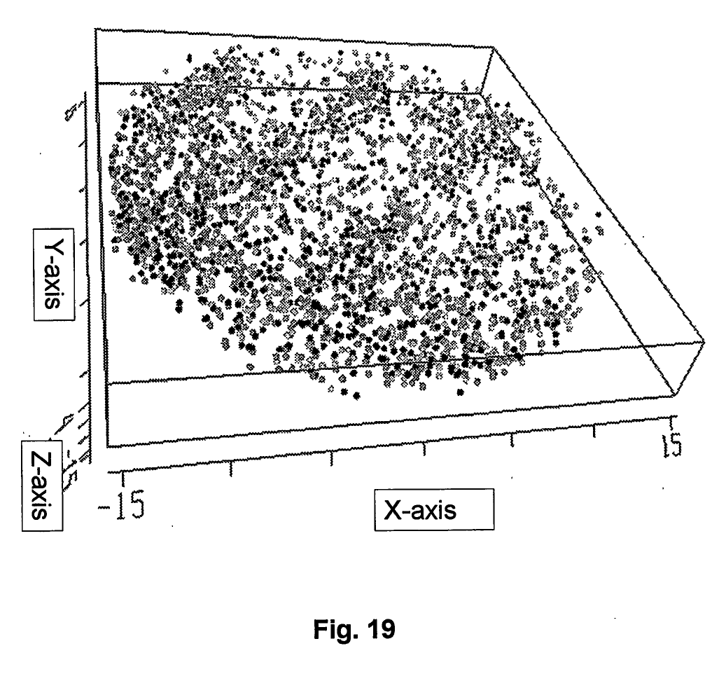

A 3-D reconstruction of the spectra shown in FIGS. 18A and 18B is provided in FIG. 19. As depicted in FIG. 19, filled black circles are carbon, grey circles are sulfur, and white circles are oxygen. Some of the atoms of the specimen are not shown in FIG. 19 for clarity purposes. The axis dimensions are in nanometers.

(15) Imaging Considerations:

Imaging proteins within embedding materials will require identifying the protein within the embedment by virtue of its differing elemental composition and structure. This can be done because the LEAP identifies all elements with atomic-scale resolution. Because the linear structure of both the embedment polymer(s) and most generally the protein are known, this can be used to facilitate this analysis. For example, the sulfur atom in the polythiophene monomer will enable ready separation of the matrix from the embedded protein because sulfur is relatively rare in proteins. In the case of proteins, the regular repeating backbone of C—C—N can also be used to highlight the protein structure. Other specimens may be readily identified by similar elemental features, such as the phosphorus in DNA and RNA. In addition, identification may be further simplified using heavy metal labels or isotopes (including deuterium) incorporated into either the protein or the ICP embedding matrix. Finally, the types of labeling that are routinely done for other methods of biomolecular structure determination may also be employed for LEAP. This includes the use of selenyl-methionine as used in x-ray crystallography, heavy element labeling for TEM, unusual elements for energy filtration (EF) TEM, and isotope labeling for NMR. For example, and as noted above, a LEAP 3-D reconstruction that shows the location of C, S and O is provided in FIG. 19.

(16) LEAP Software and Informatics:

The LEAP intrinsically determines where ions come from and what their mass/charge ratio is. From the mass/charge ratio it is possible to calculate the elemental composition of an ion, given adequate mass resolution and potentially aided by some knowledge of the material being examined. However, with respect to organics and biomolecules, understanding the connectivity of the specimen atoms (and/or molecular fragments) is important for many applications. Because the LEAP or other atom probes do not directly provide this information, computation methods are required for providing this information and for its visualization. It should be noted that the position-sensitive ion detector in the LEAP only detects about 60% of specimen ions. Therefore, within any given field of view, up to 40% of atoms (or molecular ions) within the specimen are not known. Thus, some desirable features for computational analysis include:

Algorithms to locate amino acid carbon-nitrogen “backbones” in proteins.

Algorithms to locate the ICP matrix so that it may be subtracted from the image to find the protein.

Best-fit matching of LEAP data to known bond lengths and angles.

Best-fit matching of.LEAP data to primary sequence information (when available) in order to enable calculations of missing atoms.

Algorithms to utilize data from other sources such as homology modeling, NMR, crystallography, and mass spectrometry.

Algorithms to average several structures and/or piece together multiple incomplete structures of proteins. Incomplete structures may be observed at the boundaries of an atom probe sample, or if an individual protein collapses or is entirely ionized at some time during LEAP analysis.

Software to determine the likely elemental composition of small molecular ion fragments.

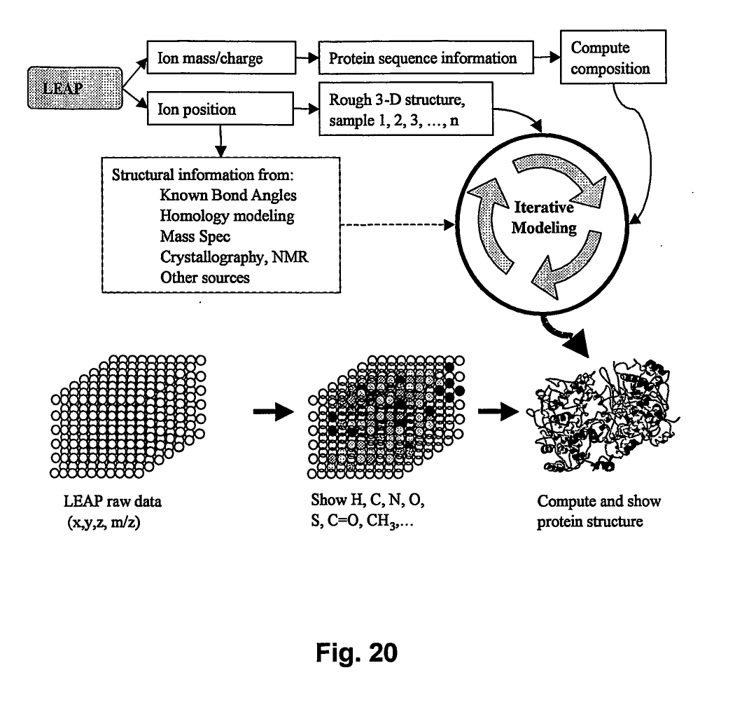

The overall approach for the analysis software is illustrated in FIG. 20. As shown in FIG. 20, the upper block diagrams illustrate the data processing pathway, including the use of information provided by other sources to assist with the analysis of the LEAP data, and the use of iteration to refine the structure. The lower figures schematically illustrate the process of extracting a protein structure from the background ICP matrix, followed by computation and display of the protein structure in the desired format (illustrated in FIG. 20 as a ribbon format). The LEAP analysis of the ICP specimens shown in FIGS. 15, 16 and 19 are equivalent to the LEAP raw data diagram shown in the lower-left corner of FIG. 20.

### BIBLIOGRAPHY

Akahori, H., M. Handa, et al. (2000). “Osmium-metal coating device using hollow-cathode plasma CVD method.” J Electron Microsc(Tokyo) 49(6): 735-744.
Albrecht, R. M., D. H. Rasmussen, et al. (1976). “Preparation of cultured cells for SEM: air drying from organic solvents.” J Microsc 108(1): 21-9.
Allen, T. D., E. M. Jack, et al. (1986). “Scanning electron microscopy of human metaphase chromosomes.” Scan Electron Microsc 1986(1): 301-8.
Auer, M. (2000). “Three-dimensional electron cryo-microscopy as a powerful structural tool in molecular medicine.” J Mol Med 78(4): 191-202.
Baker, T. S., N. H. Olson, et al. (1999). “Adding the Third Dimension to Virus Life Cycles: Three-Dimensional Reconstruction of Icosahedral Viruses from Cryo-Electron Micrographs.” Microbiol. Mol. Biol. Rev. 63(4): 862-922.
Baumeister, W. and A. C. Steven (2000). “Macromolecular electron microscopy in the era of structural genomics.” Trends Biochem Sci 25(12): 624-31.
Beesley, J. E. (1989). “Immunolabelling and electron microscopy in cytochemistry.” Curr Opin Immunol 2(6): 927-31.
Bozzola, J. J. and L. D. Russel (1999). Electron Microscopv. Principles and Techniques for Biologists. Boston, Jones and Bartlett.
Colliver, T. L., C. L. Brummel, et al. (1997). “Atomic and Molecular Imaging at the Single-Cell Level with TOF-SIMS.” Anal. Chem. 69(13): 2225 -2231.
Goldstein, J. I., D. E. Newbury, et al. (1992). Scanning Electron Microscopy and X-Ray Microanalysis: A Text for Biologists, Material Scientists, and Geologists. New York, Plenum Press.
Goodman, S. L. (1999). Scanning Electron Microscopy Evaluation of Biomaterials. Handbook of Biomaterials Evaluation: Scientific, Technical, and Clinical Testing of Implant Materials. A. von Recum. Philadelphia, Taylor and Francis: 613-629.
Goodman, S. L., C. Li, et al. (1988). Surface and bulk analysis of phase-segregation in polyurethanes by electron microscopies. The Surface Characterization of Biomaterials. B. D. Ratner. Amsterdam, Elsevier: 281-295.
Goodman, S. L., S. R. Simmons, et al. (1990). “Preferential Adsorption of Plasma Proteins onto Apolar Polyurethane Microdomains.” J Colloid Interface Sci 139: 561-570.
Heiney, P. A., K Gruneberg, et al. (2000). “Structure and growth of chromophore-functionalized (3-aminopropyl)triethoxysilane self-assembled on silicon.” Lanqmuir 16: 2651-2657.
Iwatsu, F., H. Morikawa, et al. (1987). “An attempt to image organic molecules with FIM.” J Physique C6: 263-268.
Jayasena, S. D. (1999). “Aptamers: An Emerging Class of Molecules That Rival Antibodies in Diagnostics.” Clin Chem 45(9): 1628-1650.
Kelly, T. F., P. P. Camus, et al. (1995). High Mass Resolution Local-Electrode Atom probe. USA, Wisconsin Alumni Research Foundation.
Kelly, T. F. and D. J. Larson (2000). “Local Electrode Atom Probes (Review).” Materials Characterization 44: 59-85.
Kumar, N., C. Maldarelli, et al. (2001). “Formation of nanometer domains of one chemical functionality in a continuous matrix of a second chemical functionality by sequential adsorption of silane self-assembled monolayers.” Langmuir 17: 7789-7797.
Larson, D. J., B. D. Wissman, et al. (2001). “Advances in Atom Probe Specimen Fabrication from Planer Multilayer Thin Film Structures.” Microscopy Microanalysis 7: 24-31.
Li, C., S. L. Goodman, et al. (1988). “Morphology of segmented polybutadiene-polyurethanes.” Macromolecules 21: 2367-2375.
Liu, J. and T. T. Tsong (1988). “Kinetic-energy and mass analysis of carbon cluster ions in pulsed-laser-stimulated field evaporation.” Physical Review B. Condensed Matter 38(12): 8490-8493.
Machlin, E. S., A. Freilich, et al. (1975). “Field ion microscopy of biomolecules.” J Microscopy 104:127-168.
Maruyama, T., Y. Hasegawa, et al. (1987). “FIM and Atom Probe Study of Polymers.” J Physique C6: 269-274.
Mrksich, M. and G. M. Whitesides (1996). “Using self-assembled monolayers to understand the interactions of man-made surfaces with proteins and cells.” Annual Review of Biophysics & Biomolecular Structure 25: 55-78.
Nishikawa, O., T. Sekine, et al. (1997). “Atomic investigation of individual apexes of diamond emitters by a scanning atom probe.” J Vac Sci Technol B 16(2): 836-840.
Nishikawa, O., T. Sekine, et al. (1998). “Development of a scanning atom probe and atom-by-atom mass analysis of diamonds.” Appl Phys A 66: s11-s16.
Panitz, J. A. (1981). “Point-projection imaging of macromolecular contours.” J Microscopy 125(1): 3-23.
Panitz, J. A. (1982). “point-projection imaging of unstained ferritin clusters.” Ultramicroscopy 7: 241-248.
Panitz, J. A. (1983). “Direct visualization of unstained nucleic acids on a metal substrate.” Ultramicroscopy 11: 161-166.
Prime, K L. and G. M. Whitesides (1991). “Self-assembled organic monolayers: model systems for studying adsorption of proteins at surfaces.” Science 252(5010): 1164-7.
Shiurba, R. (2001). “Freeze-substitution: origins and applications.” Int Rev Cytol 206: 45-96.
Ulman, A., Ed. (1998). Thin Films: Self-assembled monolavers of thiols. New York, Academic Press.
van Heel, M., B. Gowen, et al. (2000). “Single-particle electron cryo-microscopy: towards atomic resolution.” Q Rev Bio hvs 33(4): 307-69.
Walther, P., Y. Chen, et al. (1992). “High-resolution scanning electron microscopy of frozen-hydrated cells.” J Microsc 168(Pt 2): 169-80.
White, R. R., B. A. Sullenger, et al. (2000). “Developing aptamers into therapeutics.” J Clin Investigation 106(8): 929-934.

1. A method of preparing a specimen for microanalysis, the method comprising:
(a) embedding the specimen within an electrically conductive polymer matrix to yield an embedded specimen; and
(b) forming regions on the embedded specimen into shapes suitable for microanalysis by an atom probe.

2. The method of claim 1, wherein in step (a), the specimen is embedded within an intrinsically conductive polymer matrix.

3. The method of claim 1, wherein in step (a), the specimen is embedded within a polymer matrix and further comprising a step of treating the polymer matrix to increase its conductivity.

4. The method of claim 3, wherein the polymer matrix is treated with a metal-containing compound, wherein the treatment increases the conductivity of the polymer matrix.

5. The method of claim 3, wherein the polymer matrix is treated with a metal-containing compound selected from a group consisting of osmium-containing compounds and ruthenium-containing compounds.

6. The method of claim 3, wherein the polymer is treated with a metal-containing compound selected from a group consisting of osmium tetroxide and ruthenium tetroxide.

7. The method of claim 1, wherein in step (a), the specimen is embedded within a hydrogel.

8. The method of claim 1, wherein in step (a), the specimen is embedded within the electrically conductive polymer matrix by mixing the specimen with a corresponding monomeric compound and then polymerizing the monomeric compound to yield the electrically conductive polymer matrix.

9. The method of claim 1, wherein in step (a), the specimen is embedded within the electrically conductive polymer matrix by mixing the specimen with a corresponding pre-polymer compound and then polymerizing the pre-polymer compound to yield the electrically conductive polymer matrix.

10. The method of claim 1, wherein in step (a), the specimen is embedded within the electrically conductive polymer matrix by mixing the specimen with a corresponding water-soluble monomeric compound and then polymerizing the monomeric compound in aqueous solution to yield the electrically conductive polymer matrix.

11. The method of claim 1, wherein in step (a), the specimen is embedded within a matrix comprising a polymer selected from a group consisting of polythiophenes, polyanilines, polypyrroles, and combinations thereof.

12. The method of claim 1, wherein in step (a), the embedded specimen is disposed on a substrate prior to step (b).

13. The method of claim 1, wherein in step (a), the embedded specimen is disposed on a substrate after step (b).

14. The method of claim 1, wherein in step (b), the regions are formed using focused ion beam lithography.

15. The method of claim 1, wherein in step (b), the regions are formed by doping the embedded specimen with a masking agent and then exposing the embedded specimen to a broad ion beam under conditions and for a time sufficient to remove the masking agent from the embedded specimen, whereby regions protruding from embedded specimen and suitable for microanalysis by an atom probe are formed.

16. The method of claim 1, wherein in step (a), the embedded specimen is disposed on a substrate prior to step (b) to yield a specimen-coated substrate, and then, in step (b), forming regions on the specimen-coated substrate suitable for microanalysis by an atom probe.

17. The method of claim 16, wherein in step (b), the regions are formed using focused ion beam lithography.

18. The method of claim 1, wherein in step (b), the regions are formed by doping the specimen-coated substrate with a masking agent and then exposing the specimen-coated substrate to a broad ion beam under conditions and for a time sufficient to remove the masking agent from the embedded specimen, whereby regions protruding from embedded specimen and suitable for microanalysis by an atom probe are formed.

19. The method of claim 1, wherein in step (a), an organic or biological specimen is embedded within the matrix.

20. The method of claim 1, wherein a protein is embedded within the matrix.

21. The method of claim 1, further comprising the steps of forming regions on a substrate suitable for microanalysis by an atom probe, and immobilizing the embedded specimen on the formed regions of the substrate, whereby regions on the embedded specimen are formed into shapes suitable for microanalysis by an atom probe.

22. The method of claim 1, further comprising the steps of forming regions on a substrate suitable for microanalysis by an atom probe, and immobilizing the specimen on the formed regions of the substrate, and then coating the formed regions of the substrate with the electrically conductive matrix, whereby the specimen is embedded within the matrix.

23. The method of claim 1, further comprising stabilizing the specimen by forming internal cross-links within the specimen, by forming cross-links between the specimen and the matrix, by forming cross-links between the specimen and a substrate, or combinations thereof.

24. The method according to claim 1, further comprising step (c): and then microanalyzing the shapes formed in step (b).

25. The method of claim 24, wherein in the shapes are microanalyzed by atom probe microscopy.

26. The method of claim 24, wherein in the shapes are microanalyzed by local electrode atom probe microscopy.

27. An atom probe specimen fabricated by a method according to claim 1.

28. A composition of matter comprising an intrinsically conductive polymer whose conductivity has been altered by contact with a compound selected from a group consisting of osmium tetroxide, ruthenium tetroxide, and combinations thereof.
